(19) United States
(12) Patent Application Publication
(10) Pub. No.: US 2024/0226506 A9
(43) Pub. Date: Jul. 11, 2024

(54) DELIVERY DEVICES AND RELATED METHODS

(71) Applicant: AtriCure, Inc., Mason, OH (US)

(72) Inventors: Michael J. Banchieri, Discovery Bay, CA (US); Ara M. Stephanian, Davis, CA (US); Jeremy D. Dando, Plymouth, MN (US); Feng Liu, Pasadena, CA (US); Greg Fung, Redwood City, CA (US); Jean-Pierre Dueri, New York, NY (US)

(73) Assignee: AtriCure, Inc., Mason, OH (US)

(21) Appl. No.: 18/278,789

(22) PCT Filed: Mar. 15, 2022
(86) PCT No.: PCT/US2022/020349

§ 371 Date: Aug. 24, 2023

Related U.S. Application Data
(60) Provisional application No. 63/161,902, filed on Mar. 16, 2021.

(51) Int. Cl. A61B 17/00; A61B 90/00

(52) U.S. Cl. CPC A61B 17/00234; A61B 90/02; A61B 2017/00243; A61B 2017/00305; A61B 2017/00327; A61B 2017/00349; A61B 2017/00367; A61B 2017/00557; A61B 2017/00982; A61B 2217/005

(57) ABSTRACT

Delivery devices configured to deliver therapeutic and visualization devices to a surgical site are disclosed. An example delivery device may include a proximally disposed handle; a shaft extending distally from the handle, the shaft including a therapeutic device lumen configured to deliver a working end of a therapeutic device therethrough, a first portion of the shaft being steerable; and a space-making element disposed proximate a distal end portion of the shaft and configured to separate biological tissues to create or expand a working space. At least a portion of the space-making element may be reconfigurable between a retracted configuration and an expanded configuration.

### CROSS REFERENCE TO RELATED APPLICATIONS

This application claims the benefit of U.S. Provisional Application No. 63/161,902, filed Mar. 16, 2021, which is incorporated by reference.

### INTRODUCTION

The present disclosure is directed to medical instruments and devices for conducting surgical procedures, and, more specifically, to delivery devices, such as cannulas, for surgical procedures, and related methods.

The present disclosure contemplates that some medical procedures, such as minimally invasive surgical procedures, may involve the use of elongated surgical instruments at a surgical site within a patient's body. In some such procedures, it may be advantageous to route the surgical instruments to the surgical site along a path that curves around anatomical structures, such as to minimize trauma or other impact to anatomical structures interposing the skin access point and the surgical site. For example, minimally invasive procedures involving some portions of a patient's heart (e.g., the left atrial appendage) may involve a skin penetration on the front of the patient's abdomen, a penetration through the pericardium, and a path along the posterior aspect of the patient's heart. Because such a path may not be straight, it may be difficult to route various surgical instruments to the surgical site.

While known devices and techniques have been used to perform minimally invasive procedures, improvements in the construction and operation of delivery devices for surgical instruments may be beneficial for users (e.g., surgeons) and patients. The present disclosure includes various improvements which may enhance the construction, operation, and methods of use of delivery devices, such as for minimally invasive procedures.

It is an aspect of the present disclosure to provide a delivery device configured to deliver a therapeutic device to a surgical site, the delivery device including a proximally disposed handle, an elongated shaft extending distally from the handle, the shaft including a generally longitudinal therapeutic device lumen configured to deliver a working end of a therapeutic device therethrough, and/or a space-making element disposed proximate a distal end portion of the shaft and configured to separate biological tissues to create or expand a working space. A first portion of the shaft may be steerable and/or at least a portion of the space-making element may be reconfigurable between a retracted configuration and an expanded configuration.

In a detailed embodiment, the shaft may include a generally longitudinal visualization device lumen configured to deliver a working end of a visualization device therethrough. The handle may include a proximal port operatively connected to the therapeutic device lumen and the visualization device lumen and/or the proximal port may be configured to receive the therapeutic device and/or the visualization device therethrough.

In a detailed embodiment, the shaft may include a first generally longitudinal suction lumen configured to facilitate application of suction proximate the distal end portion of the shaft. The shaft may include a second generally longitudinal suction lumen configured to facilitate application of suction proximate the distal end portion of the shaft.

In a detailed embodiment, in the retracted configuration, the space-making element may have a width about the same as a width of the shaft. In the expanded configuration, the space-making element may have a width substantially greater than the width of the shaft.

In a detailed embodiment, in the retracted configuration, at least a portion of the space-making element may be disposed generally concentrically within the distal end portion of the shaft. The distal end portion of the shaft may include a recess configured to receive at least a portion of the space-making element in the retracted configuration.

In a detailed embodiment, the space-making element may include an inflatable space-making element. The shaft may include a first generally longitudinal inflation lumen operatively coupled to supply inflation fluid to the inflatable space-making element. The inflatable space-making element may be generally in the form of a right circular cylinder. The inflatable space-making element may include a plurality of generally circumferentially oriented ribs. The inflatable space-making element may include a plurality of generally longitudinally oriented ribs. The inflatable space-making element may include at least one orientation feature. The orientation feature may include a notch. The inflatable space-making element may include a distal face that is angled relative to a plane normal to a longitudinal axis of the shaft. The inflatable space-making element may include a plurality of longitudinally oriented inflatable elements. At least one of the plurality of longitudinally oriented inflatable elements may be in the form of a generally circular tube. At least one of the plurality of longitudinally oriented inflatable elements may be in the form of a tube having a generally oval cross section. The inflatable space-making element may include at least one collapsible structural element configured to facilitate spacing of the longitudinally oriented inflatable elements. The collapsible structural element may include a collapsible nitinol frame. The inflatable space-making element may include at least one generally helical inflatable element. The inflatable space-making element may include two generally helical inflatable elements in an alternating arrangement.

In a detailed embodiment, the first portion of the shaft may be steerable in a first plane. The handle may include a steering actuator configured to steer the first portion of the shaft in the first plane. The first portion of the shaft may be steerable uni-directionally in the first plane. The first portion of the shaft may be steerable bi-directionally in the first plane. The first portion of the shaft may be deformable in a second plane. The second plane may be generally perpendicular to the first plane. The first portion of the shaft may be flexible in the second plane. The first portion of the shaft may be steerable in the second plane. The first portion of the shaft may be steerable bi-directionally in the first plane and/or bi-directionally in the second plane. The delivery device may include a first steering wire operatively coupling the steering actuator and the first portion of the shaft for steering the first portion of the shaft a first direction in the first plane. The delivery device may include a second steering wire operatively coupling the steering actuator and the first portion of the shaft for steering the first portion of the shaft in a second direction in the first plane, the second direction generally opposite the first direction. A second portion of the shaft may be flexible in a second plane. The second plane may be generally perpendicular to the first plane. The handle may include at least one locking actuator configured to selectively lock the steering actuator.

In a detailed embodiment, a second portion of the shaft may be flexible in a first plane. The second portion of the shaft may be flexible in a second plane. The second plane may be generally perpendicular to the first plane. A flexibility of the second portion of the shaft in the first plane may be greater than a flexibility of the second portion of the shaft in the second plane. A stiffness of the second portion of the shaft in the first plane may be greater than a stiffness of the second portion of the shaft in the second plane. The second portion of the shaft may be substantially uniformly flexible over a longitudinal length of the second portion of the shaft. A shaft flexibility may vary longitudinally over a longitudinal length of the second portion of the shaft. A flexibility of the first portion of the shaft may differ from a flexibility of the second portion of the shaft.

In a detailed embodiment, the shaft may include an outer jacket and a structural component disposed radially within the outer jacket. The structural component may include a hypotube. The hypotube may include a plurality of solid portions and/or a plurality of gaps configured to facilitate desired flexibility characteristics of the shaft. The hypotube may include a first arrangement of the solid portions and/or the gaps in the first portion of the shaft and/or a second arrangement of the solid portions and/or the gaps in a second portion of the shaft so that the first portion of the shaft and the second portion of the shaft have different flexibility characteristics.

In a detailed embodiment, the delivery device may include an obturator positioned distally on the shaft. The obturator may be inflatable. The obturator may be selectively removable from the delivery device.

In a detailed embodiment, the therapeutic device may include at least one of an ablation device, a biopsy device, a pericardial lead, a needle for delivering a therapeutic agent, and/or a surgical tool.

In a detailed embodiment, the visualization device may include an endoscope.

It is an aspect of the present disclosure to provide a delivery device configured to deliver a therapeutic device to a surgical site, the delivery device including: a proximally disposed handle, an elongated shaft extending from the handle, the shaft comprising a generally longitudinal therapeutic device lumen configured to deliver a working end of a therapeutic device therethrough, and/or a space-making element disposed proximate a distal end portion of the shaft and configured to separate biological tissues to create or expand a working space. A first portion of the shaft may be flexible.

In a detailed embodiment, the space-making element may be reconfigurable between a retracted configuration and an expanded configuration by inflation and deflation of at least one inflatable element.

In a detailed embodiment, the space-making element may be substantially rigid. The space-making element may include a deep, angled hood.

In a detailed embodiment, the first portion of the shaft may be flexible in a first plane. A second portion of the shaft may be flexible in a second plane. The second plane may be generally perpendicular to the first plane. The first portion of the shaft may include a distal portion. The second portion of the shaft may include a proximal portion. The first portion of the shaft may be steerable in the first plane by operation of a steering actuator disposed on the handle. The first portion of the shaft may be flexible in the second plane.

It is an aspect of the present disclosure to provide a method of using a delivery device, including: positioning a distal end portion of an elongated shaft of a delivery device at a desired location; expanding a reconfigurable space-making element disposed distally on the shaft from a retracted configuration to an expanded configuration; creating or expanding a working space at the desired location using the space-making element; and/or directing a working end of a therapeutic device to the desired location through the shaft via a longitudinally extending therapeutic device lumen.

In a detailed embodiment, positioning the distal end portion of the elongated shaft of the delivery device at the desired location may include advancing the shaft using a handle disposed proximally on the shaft. The distal end portion of the shaft may be steerable by a steering actuator disposed on the handle. Positioning the distal end portion of the shaft of the delivery device at the desired location may include operating the steering actuator to steer the distal end portion of the shaft. Operating the steering actuator to steer the distal end portion of the shaft may include pulling a steering wire extending longitudinally through the shaft to deflect of the distal end portion.

In a detailed embodiment, a proximal portion of the shaft may be flexible in a first plane. Positioning the distal end portion of the shaft of the delivery device at the desired location may include bending the proximal portion of the shaft in the first plane. The distal end portion of the shaft may be steerable in a second plane. The second plane may be generally perpendicular to the first plane. Positioning the distal end portion of the shaft of the delivery device at the desired location comprises steering the distal end portion of the shaft in the second plane. The distal end portion of the shaft may be flexible in the first plane. Positioning the distal end portion of the shaft of the delivery device at the desired location may include bending the distal end portion of the shaft in the first plane.

In a detailed embodiment, the reconfigurable space-making element may include an inflatable space-making element. Expanding the reconfigurable space-making element may include delivering an inflation fluid to the inflatable space-making element through a longitudinally disposed inflation lumen in the shaft.

In a detailed embodiment, expanding the reconfigurable space-making element may include laterally expanding the reconfigurable space-making element.

In a detailed embodiment, expanding the reconfigurable space-making element may include longitudinally expanding the reconfigurable space-making element.

In a detailed embodiment, positioning the distal end portion of the elongated shaft of the delivery device at the desired location may include positioning the distal end portion proximate a left atrial appendage of a patient's heart. Positioning the distal end portion proximate the left atrial appendage may include advancing the distal end portion posterior to the patient's heart.

It is an aspect of the present disclosure to provide any apparatus, method, or combination thereof as disclosed herein. It is an aspect of the present disclosure to provide any two or more of the foregoing aspects and/or features in any combination. It is an aspect of the present disclosure to provide any combination of elements from one or more of the foregoing aspects and/or features.

### BRIEF DESCRIPTION OF THE DRAWINGS

Example embodiments are described in conjunction with the accompanying drawing figures in which:

FIG. 1 is a simplified schematic view of an example delivery device;

FIG. 2 is a perspective view of an example delivery device;

FIG. 3 is a simplified cross-sectional view of an example delivery device;

FIG. 4 is a perspective view of an example handle of a delivery device;

FIG. 5A is a distal perspective view of an example space-making element generally in the form of a right cylinder;

FIG. 5B is a distal perspective view of an example space-making element generally in the form of a deep angled hood;

FIG. 5C is a distal perspective view of an example space-making element generally in the form of a shallow angled hood;

FIG. 5D is a distal perspective view of an example space-making element generally in the form of a deep angled hood;

FIG. 5E is a distal perspective view of an example space-making element generally in the form of a shaped distal end portion of a shaft;

FIG. 5F is a distal perspective view of an example space-making element generally in the form of a flat distal end face of a shaft;

FIG. 5G is a perspective view of an example space-making element generally in the form of an extended cylinder with an angled tip;

FIG. 6A is a simplified cross-sectional view of a distal portion of a delivery device including an example inflatable space-making element in a retracted configuration;

FIG. 6B is a simplified cross-sectional view of the inflatable space-making element of FIG. 6A in an expanded configuration;

FIG. 6C is a simplified side view of a distal portion of a delivery device including an example inflatable space-making element in a deflated configuration and inflatable obturator in an inflated configuration;

FIG. 6D is a simplified cross-sectional view of the distal portion of the delivery device of FIG. 6C with the inflatable space-making element in an inflated configuration;

FIG. 7A is a simplified cross-sectional view of a distal portion of an example delivery device including expandable space-making elements disposed proximally from where a scope emerges;

FIG. 7B is a distal view of the delivery device of FIG. 7A;

FIG. 8 is a distal perspective view of an example delivery device including a selectively deployable skirt;

FIG. 9 is a perspective view of a distal portion of an example delivery device including a movable space-making element;

FIG. 10 is a simplified side view of a distal portion of an example delivery device including a movable space-making element;

FIG. 11 is a simplified side view of a distal portion of an example delivery device including a movable space-making element;

FIG. 12A is a simplified side view of a distal portion of an example delivery device including two expandable elements;

FIG. 12B is a distal view of the delivery device of FIG. 12A;

FIG. 13A is a simplified cross-sectional view of an example shaft including a rotatable inner shaft portion;

FIG. 13B is a perspective view of the distal end portion of the shaft of FIG. 13A;

FIG. 14 is a simplified cross-sectional view of an example shaft including a rotatable outer shaft portion;

FIG. 15A is a simplified cross-sectional view of an example shaft including an outer shaft portion having a non-circular outer perimeter;

FIG. 15B is a perspective view of the distal end portion of the shaft of FIG. 15A;

FIG. 16 is a simplified cross-sectional view of a delivery device including a lock-brake mechanism and an endoscope;

FIG. 17 is a top view of an alternative example delivery device;

FIG. 18 is a proximal perspective view of the delivery device of FIG. 17;

FIG. 19 is a detailed distal perspective view of the delivery device of FIG. 17;

FIG. 20 is a detailed distal perspective cross-section view of the shaft of the delivery device of FIG. 17;

FIG. 21 is a proximal perspective view of a structural component of the shaft of the delivery device of FIG. 17;

FIG. 22 is a detailed perspective view of a distal end portion of the structural component of the shaft of the delivery device of FIG. 17;

FIG. 23 is a proximal perspective view of an alternative example inflatable space-making element including circumferential ribs;

FIG. 24 is a proximal perspective view of an alternative example inflatable space-making element including longitudinal ribs;

FIG. 25 is a distal perspective view of an alternative example inflatable space-making element including an orientation feature;

FIG. 26 is a side view of an alternative example inflatable space-making element including an angled distal face;

FIG. 27 is a simplified side cross-section view of an alternative inflatable space-making element including a plurality of longitudinally orientated inflatable elements;

FIG. 28 is a simplified distal end view of the inflatable space-making element of FIG. 27;

FIG. 29 is a simplified side cross-section view of an alternative inflatable space-making element including a plurality of flattened longitudinally orientated inflatable elements;

FIG. 30 is a simplified distal end view of the inflatable space-making element of FIG. 29;

FIG. 31 is a simplified side view of an alternative inflatable space-making element including coiled inflatable elements;

FIG. 32 is a simplified distal end view of the inflatable space-making element of FIG. 31;

FIG. 33 is a distal perspective view of a distal end portion of an alternative example delivery device including a rigid space-making element;

FIG. 34 is a detailed perspective view of a distal end portion of an alternative example structural component of a shaft of a delivery device;

FIG. 35 is a top view of an alternative example delivery device;

FIG. 36 is a perspective view of alternative example delivery devices;

FIG. 37 is a perspective view of an alternative example delivery device;

FIG. 38 is a perspective view of alternative example delivery devices;

FIG. 39 is a perspective view of alternative example delivery devices; all in accordance with at least some aspects of the present disclosure.

### DETAILED DESCRIPTION

Example embodiments according to the present disclosure are described and illustrated below to encompass devices, methods, and techniques relating to medical procedures. Of course, it will be apparent to those of ordinary skill in the art that the embodiments discussed below are examples and may be reconfigured without departing from the scope and spirit of the present disclosure. It is also to be understood that variations of the example embodiments contemplated by one of ordinary skill in the art shall concurrently comprise part of the instant disclosure. However, for clarity and precision, the example embodiments as discussed below may include optional steps, methods, and features that one of ordinary skill should recognize as not being a requisite to fall within the scope of the present disclosure.

The present disclosure includes, among other things, medical instruments and devices for conducting surgical procedures and, more specifically, delivery devices, such as cannulas, for surgical procedures, and related methods. Some example embodiments according to at least some aspects of the present disclosure may be useful in connection with minimally invasive procedures. For example, some example embodiments may be used in connection with ablation of cardiac tissue, such as to treat cardiac arrhythmias like atrial fibrillation.

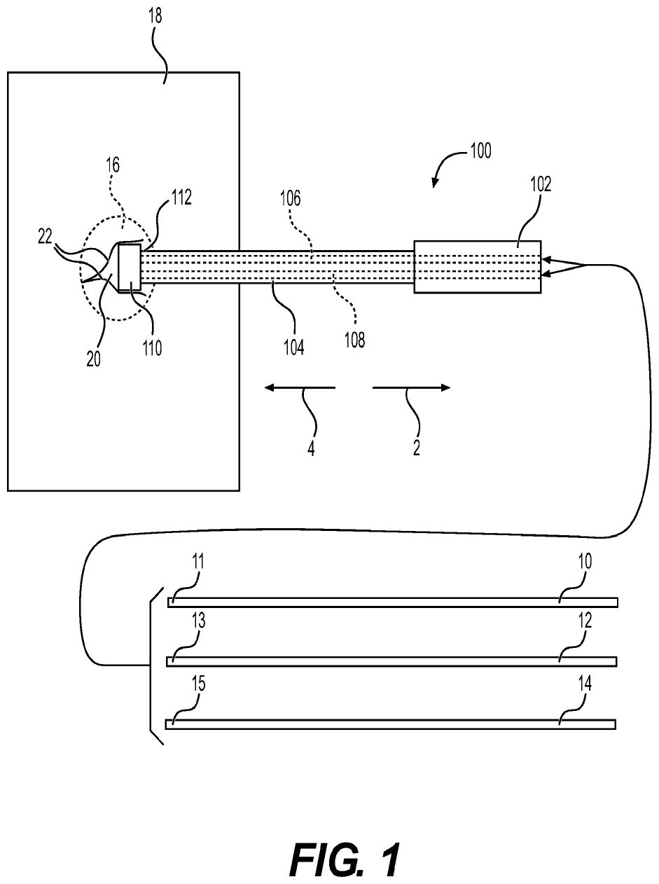

FIG. 1 is a simplified schematic view of an example delivery device 100, according to at least some aspects of the present disclosure. Some example delivery devices 100 may be configured for use in surgical procedures, such as to deliver one or more therapeutic devices 10, 12 and/or one or more visualization devices 14 to a surgical site 16 in a patient's body 18. Example therapeutic devices 10, 12 may include ablation devices, biopsy devices, pericardial leads, needles for delivering therapeutic agents, surgical tools, etc. Example visualization devices 14 may include endoscopes and other similar instruments. In some example embodiments, a separate visualization device 14 may be replaced by a chip-on-tip feature, which may comprise a digital imaging device disposed generally distally on the delivery device 100, such as generally where an endoscope would emerge.

In some example embodiments, the delivery device 100 may include a handle 102, which may be disposed proximally and/or which may generally remain outside of the patient's body 18. As used herein, “proximal” may refer to a direction generally toward an operator of a system or device (e.g., a cardiac surgeon or interventional cardiologist), such as away from the distant-most end of a device that is inserted into a patient's body. The proximal direction is indicated by arrow 2 in the drawings. The delivery device 100 may include an elongated shaft 104, which may extend distally from the handle 102. As used herein, “distal” may refer to a direction generally away from an operator of a system or device (e.g., a cardiac surgeon or interventional cardiologist), such as toward the distant-most end of a device that is inserted into a patient's body. The distal direction is indicated by arrow 4 in the drawings. At least a portion of the shaft 104 may be inserted into the patient's body 18, such as to proximate the surgical site 16. The handle 102 and/or the shaft 104 may include one or more generally longitudinal lumens 106, 108 extending therethrough. The working ends 11, 13, 15 of therapeutic devices 10, 12 and/or visualization components 14 may be delivered to the surgical site through one or more of the lumens 106, 108.

Some example embodiments may include one or more space-making elements 110, which may be disposed proximate a distal end portion 112 of the shaft 104. In various example embodiments, space-making elements 110 may be configured to create or expand a working space 20, such as by separating biological tissues 22 at the surgical site 16, which may facilitate visualization of the surgical site 16, such as by one or more visualization devices 14, and/or use of one or more therapeutic devices 10,12 at the surgical site 16. For example, use of a space-making element 110 may facilitate positioning of the therapeutic devices 10, 12 and/or may facilitate visualization of the patient's anatomy and/or the therapeutic devices 10, 12 using the visualization device 14. Some space-making elements 110 may be configured with a low profile to facilitate introduction/maneuverability into tight spaces and/or may be configured to be substantially atraumatic in various expected use conditions. In some example embodiments, a space-making element 110 may fixedly and/or rigidly disposed proximate the distal end portion 112 of the shaft 104. In some example embodiments, space-making elements 110 may be movably and/or reconfigurably disposed proximate the distal end portion 112 of the shaft 104.

The foregoing description of the example delivery device 100 provides context for various example embodiments described below. The various example embodiments described below may comprise components of the example delivery device 100, or may be utilized in alternative apparatus. The descriptions below of various example embodiments may focus on the differences between various example configurations. Description of aspects or features common among several embodiments is not repeated for brevity. Accordingly, it should be understood that various aspects, features, and characteristics described in connection with any embodiment may also pertain to other embodiments, whether or not explicitly noted below. More generally, any feature or component of any example embodiment described herein may be utilized in connection with any other embodiment, and repeated description of similar components is omitted for brevity.

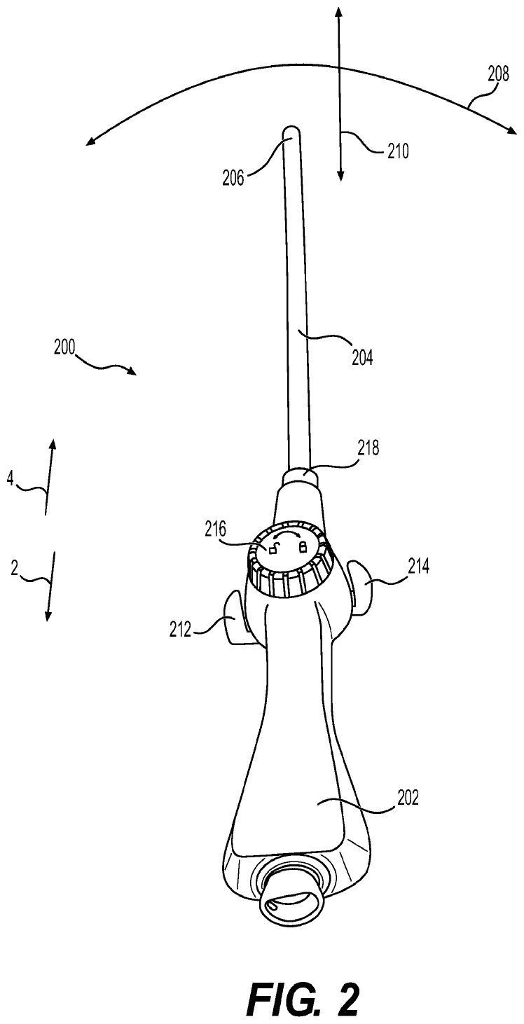

FIG. 2 is a perspective view of an example delivery device 200, according to at least some aspects of the present disclosure. Delivery device 200 may include a handle 202 and a shaft 204. In some example embodiments, any portion(s) of the shaft 204 (e.g., a distal end portion 206 of the shaft 204) may be steerable in one or more planes 208, 210, which may be generally perpendicular. As used herein, “steerable” in the context of a shaft may describe a deformable shaft (or portion thereof) that may be actively steered (e.g., bent or articulated) using an actuator. As used herein, “deformable” in the context of a shaft may describe a shaft that is configured to change shape when subject to forces expected during normal operation of the delivery device. Some example embodiments may be steerable uni-directionally or bi-directionally in each plane. Some uni-directionally steerable embodiments may be steerable in one direction from a generally central position in a particular plane (e.g., left, but not to the right, and/or down, but not up). Some embodiments may be steerable bi-directionally (e.g., left and right and/or up and down). Some example embodiments may be steerable bi-directionally in two generally perpendicular planes (e.g., left-right and up-down). In some example embodiments, steerability in one or more planes may be symmetrical or asymmetrical. For example, some asymmetrically steerable embodiments may be steerable in one direction (e.g., left) to a different extent and/or in a different curvature than in the opposite direction (e.g., right).

Some example embodiments may include one or more actuators 212, 214 configured to steer one or more portions of the shaft, such as the distal end portion 206. For example, the actuators 212, 214 may be disposed on the handle 202 for operation by a user. In some example embodiments, the actuators 212, 214 may be operatively coupled to the distal end portion 206 by one or more wires, which may facilitate steering of the distal end portion 206. Some example embodiments may include 1-4 pull wires to actuate deflection of the distal end portion 206, for example. Some example embodiments may include one or more actuators 216 configured to rotate certain components of the delivery device 200. Some example embodiments may include one or more strain relief elements, such as a strain relief element 218 proximate an interface between the handle 202 and the shaft 204.

In some example embodiments, any portion(s) of the shaft 204 (e.g., a distal end portion 206 of the shaft 204) may be deformable, such as flexible. As used herein, “flexible” in the context of a shaft may describe a shaft that is readily passively bendable (elastically and/or plastically) by application of forces expected during normal operation directly to the shaft. For example, a user may apply flexing or bending forces directly to the shaft 204, or the anatomy through which the shaft 204 is directed may apply flexing or bending forces directly to the shaft 204. In contrast, as used herein, “rigid” in the context of a shaft may describe a shaft that is not readily significantly deformable when subject to forces expected during normal operation. In various example embodiments, the shaft 204 may be flexible uni-directionally and/or bi-directionally, and symmetrically and/or asymmetrically, as described above regarding steerability.

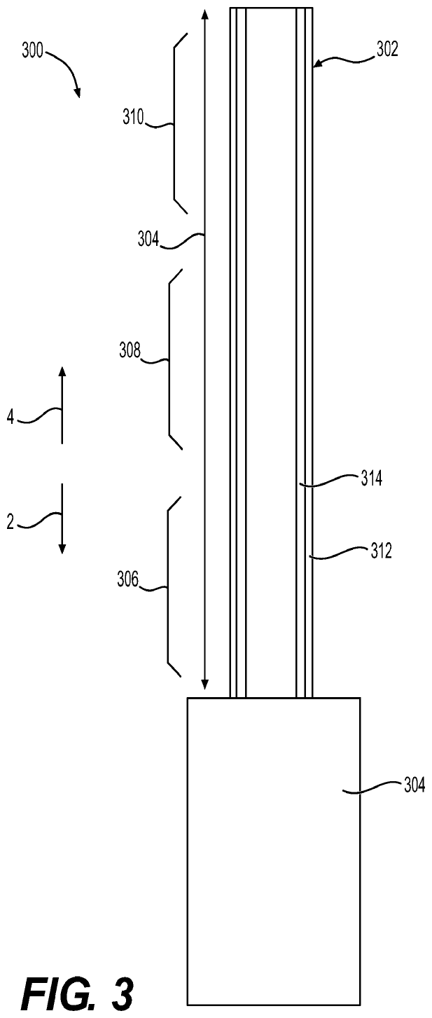

FIG. 3 is a simplified cross-sectional view of an example delivery device 300, according to at least some aspects of the present disclosure. Delivery device 300 may comprise a shaft 302 disposed distally on a handle 304. In the illustrated embodiment, the shaft 302 may be generally flexible. In some example embodiments, the shaft 302 may have substantially uniform flexibility (or stiffness) over its longitudinal length 304.

In some example embodiments, the flexibility (or stiffness) of the shaft 302 may vary longitudinally along the shaft. For example, the shaft 302 may include a plurality of zones (or portions) having different flexibilities, such as a proximal zone 306, an intermediate zone 308, and/or a distal zone 310. In some example embodiments, the shaft 302 may be relatively stiffer in the proximal zone 306, may be relatively softer in the intermediate zone 308, and/or may be most flexible in the distal zone 310. Some example embodiments may include discrete zones 306, 308, 310 with apparent and/or abutting boundaries. Other example embodiments may include spaced apart and/or gradual transitions between zones 306, 308, 310, or even a gradual variation along all or substantial portions of the shaft 302.

In some example embodiments, varying flexibility (or stiffness) over the length 304 of the shaft 302 may be facilitated by varying a thickness of a tubular component comprising the shaft 302 along its length 304. For example, the shaft 302 may comprise a polymer over jacket 312. The over jacket 312 may be thickest in the proximal zone 306, thinner in the intermediate zone 308, and/or thinnest in the distal zone 310.

In some example embodiments, varying flexibility (or stiffness) over the length 304 of the shaft 302 may be facilitated by varying the mechanical properties of the over jacket 312 along its length 304. For example, the over jacket 312 may have a relatively hard durometer in the proximal zone 306, a softer durometer in the intermediate zone 308, and a relatively soft durometer in the distal zone 310.

In some example embodiments, varying flexibility (or stiffness) over the length 304 of the shaft 302 may be facilitated by varying the construction of a structural component 314 of the shaft 302 along its length 304. For example, a shaft 302 may include braided wire as a structural component 314 (e.g., about 16 wires arranged in a crisscross (braided) pattern). In some embodiments, the braided wire structural component may be generally in the form of a braided tube. The braid pattern and/or the tightness of the weave may vary along the length to provide different shaft 302 flexibility (or stiffness). As another example, a shaft 302 may comprise a tube having a pattern laser cut therein as a structural component 314. The laser cut pattern may vary along the length 304 of the shaft 302 to provide the desired flexibility (or stiffness) characteristics.

In some example embodiments, a steerable shaft may be configured similarly to shaft 302. That is, the steerability of the shaft may vary over the length of the shaft, generally as described above with reference to FIG. 3.

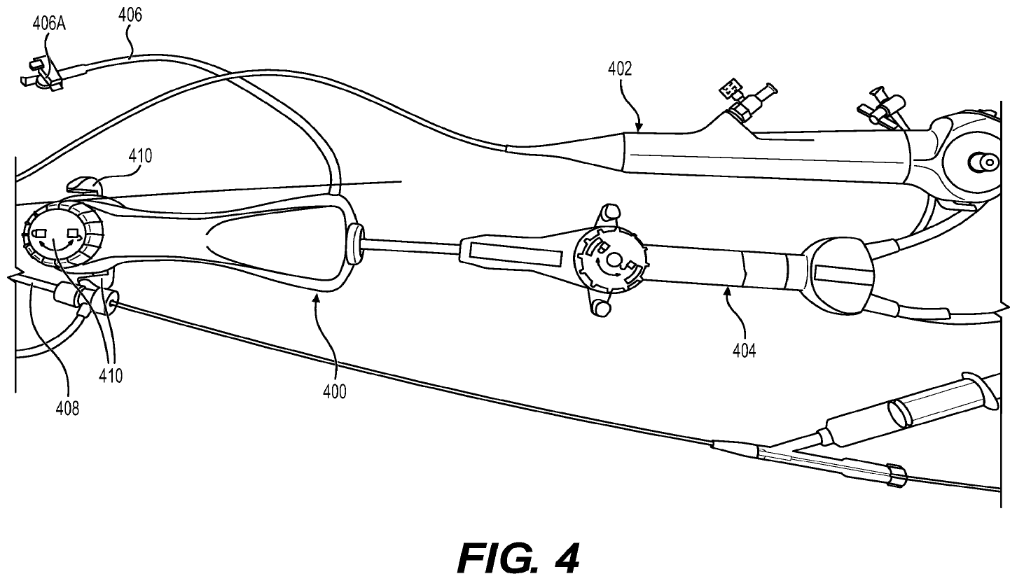

FIG. 4 is a perspective view of an example handle 400 of an example delivery device, according to at least some aspects of the present disclosure. In FIG. 4, the handle 400 is shown in use with a visualization device, such as an endoscope 402, and a therapeutic device 404. The visualization device may be received in a minor lumen of the delivery device and/or the therapeutic device 404 may be received in a main lumen of the delivery device. Also shown is a lumen 406 and a connector 406A operatively connected to supply and/or remove inflation fluid (e.g., gas and/or liquid) from one or more space-making elements comprising an inflatable element via a lumen of the delivery device. In some example embodiments, lumen 406 may be connected to a vacuum source to provide aspiration functionality. In some example embodiments, lumen 406 may be provided with a stopcock operable to allow selection between fluid injection and vacuum aspiration. Also shown is a lumen 408 configured for fluid management, such as supply or removal of fluids proximate the surgical site 16 via a lumen of the delivery device. In this example embodiment, the handle 400 may comprise a plurality of actuators 410, which may be operative to steer one or more portions of the shaft of the delivery device via a deflection mechanism, which may be at least partially disposed within the handle. Some example handles 400 may be shaped in a generally ergonomic manner.

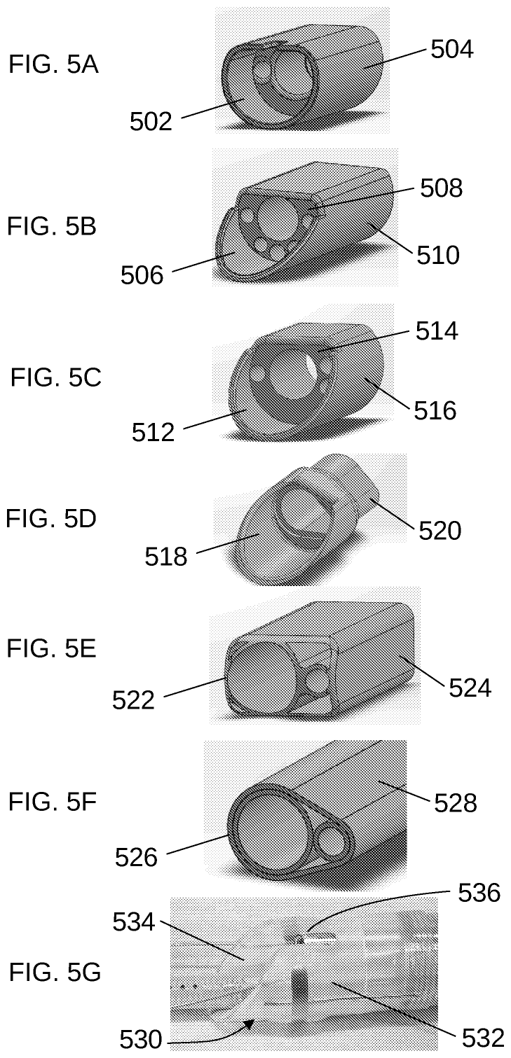

FIGS. 5A-5G are distal perspective views of various alternative example rigid and/or fixed space-making elements, which may be disposed distally on delivery devices, all according to at least some aspects of the present disclosure. As illustrated in FIGS. 5A-5G, various space-making elements may include openings associated with the lumens in the delivery device (e.g., lumens for therapeutic devices, lumens for movable space-making element such as inflatable elements, suction lumens, and/or lumens for visualization devices). FIG. 5A illustrates a space-making element 502 generally in the form of a right cylinder having a cross-sectional shape generally matching that of the shaft 504. FIG. 5B illustrates a space-making element 506 generally in the form of a deep angled hood extending from only a portion of the distal end face 508 of the shaft 510. FIG. 5C illustrates a space-making element 512 generally in the form of a shallow angled hood extending from only a portion of the distal end face 514 of the shaft 516. FIG. 5D illustrates a space-making element 518 generally in the form of a deep angled hood having a shape that differs from the shape of the shaft 520. FIG. 5E illustrates a space-making element 522 generally in the form of a shaped distal end portion of the shaft 524. FIG. 5F illustrates a space-making element 526 generally in the form of a flat distal end face of the shaft 528. In some example embodiments, a distal end portion, such as a flat distal end face, may have a diameter relatively larger than that of a proximal component (e.g., shaft). The difference in relative diameters may act as a space-making element to provide better viewing at the surgical site. FIG. 5G illustrates a space-making element 530 generally in the form of an extended cylinder 532 with an angled tip 534. Additionally, the illustrated embodiment includes a longitudinal slot disposed proximate the short aspect of the angled tip.

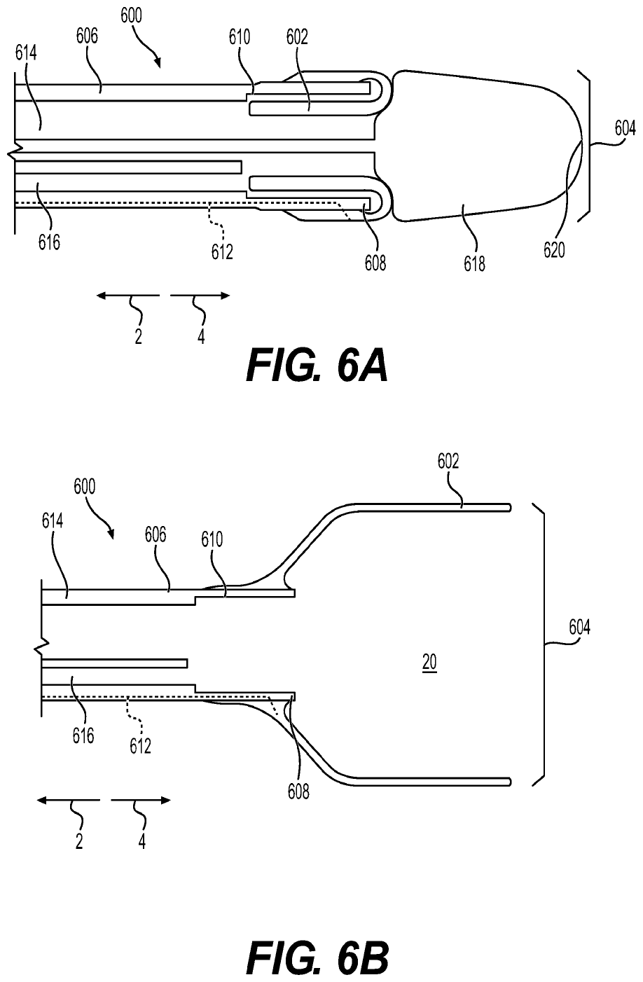

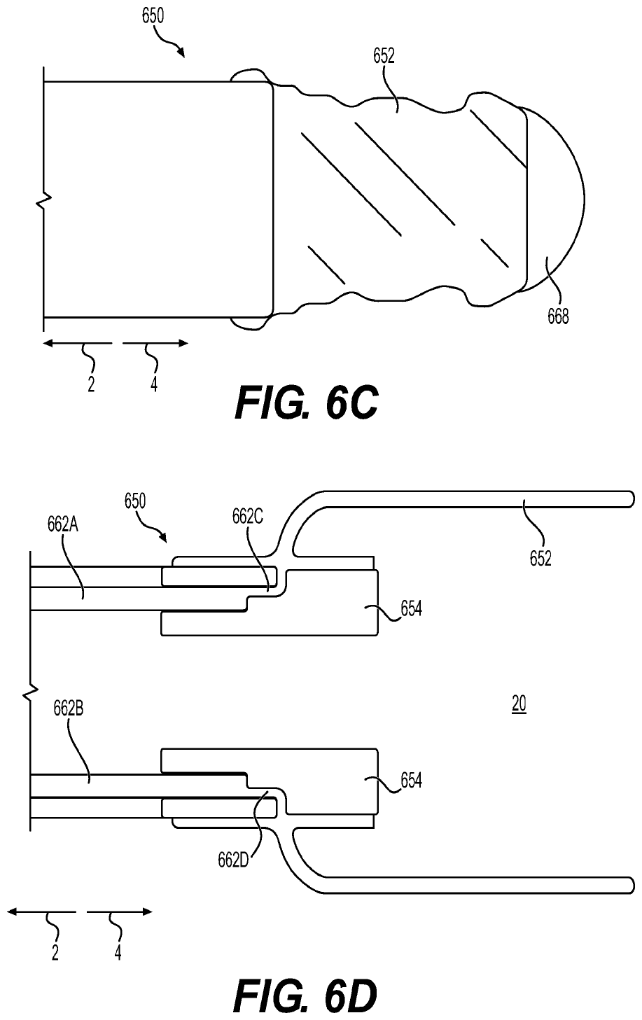

FIGS. 6A and 6B are simplified cross-sectional views of a distal portion of a delivery device 600 comprising an example inflatable space-making element 602, all according to at least some aspects of the present disclosure. Generally, the inflatable space-making element 602 may be reconfigurable between a retracted (deflated) configuration (FIG. 6A) and an expanded (inflated and/or deployed) configuration (FIG. 6B). The inflatable space-making element 602 may be used to create or expand a working space 20.

In some example embodiments, in the retracted configuration, the inflatable space-making element 602 may have a width 604 (e.g., diameter) about the same as the width (e.g., diameter) of the shaft 606. In the expanded configuration, the inflatable space-making element 602 may have a width 604 substantially greater than the width (e.g., diameter) of the shaft 606. The inflatable space-making element 602 may be laterally and/or radially expandable. In some example embodiments, in the retracted configuration, the inflatable space-making element 602 may not extend substantially distally beyond the distal end portion 608 of the shaft 606. In some example embodiments, in the expanded configuration, the inflatable space-making element 602 may extend substantially distally beyond the distal end portion 608 of the shaft 606. The inflatable space-making element 602 may be longitudinally and/or axially expandable. In some example embodiments, the inflatable space-making element 602 may be laterally/radially expandable and/or longitudinally/axially expandable.

In some example embodiments, in the retracted configuration, the inflatable space-making element 602 may be disposed generally concentrically within the distal end portion 608 of the shaft 606, such as by collapsing and/or folding generally radially inward and/or proximally. The interior of the distal end portion 608 of the shaft 606 may include a generally circumferential recess 610, which may receive at least a portion of the retracted inflatable space-making element 602.

In some example embodiments, a fluid (e.g., gas and/or liquid) may be supplied to and/or removed from the inflatable space-making element 602 via a lumen 612, which may be referred to as an inflation lumen. Example delivery devices may include other lumens, such as a device lumen 614 (e.g., for therapeutic devices 10, 12), a scope lumen 616 (e.g., for a visualization device 14), and/or a suction lumen.

Some example embodiments may include a distally positioned obturator, such as an inflatable obturator 618. In some example embodiments, the inflatable obturator 618 may include a generally rounded and/or generally blunt distal tip portion 620. In some example embodiments, the inflatable obturator 618 may be positioned proximate the distal end portion 608 of the shaft 606 via a lumen, such as the device lumen 614. In use, the delivery device 600 may be directed to and/or positioned at the surgical site with the obturator 618 positioned distally on the shaft as shown in FIG. 6A. Then, the obturator 618 may be deflated and/or withdrawn proximally, leaving the other portions of the delivery device 600 in place as shown in FIG. 6B. In some example embodiments comprising a selectively removable obturator 618, the obturator 618 may be removed from the delivery device 600 and/or the lumen 614 may be utilized to deliver other instruments (e.g., therapeutic devices) to the surgical site.

FIG. 6C is a simplified side view of a distal portion of a delivery device 650 comprising an example inflatable space-making element 652 in a deflated configuration and inflatable obturator 668 in an inflated configuration, and FIG. 6D is a simplified cross-sectional view of the distal portion of the delivery device 650 with the inflatable space-making element 652 in an inflated configuration, all according to at least some aspects of the present disclosure. Generally, the delivery device 650, inflatable space-making element 652, and obturator 668 may be similar to the delivery device 600, inflatable space-making element 602, and obturator 618 described above with reference to FIGS. 6A and 6B, and repeated description of similar elements is omitted for brevity. In the embodiment of FIGS. 6C and 6D, however, the inflatable space-making element 652 may be configured to collapse generally radially inwardly, such as generally radially inwardly against the obturator 668. That is, in the illustrated embodiment, in the retracted/deflated configuration, this inflatable space-making element 652 may not be collapsible and/or foldable generally concentrically within the distal end portion of the shaft by collapsing generally radially inwardly and proximally.

Referring to FIG. 6D, the delivery device 650 may include a distal cap 654, which may be configured to organize the various lumens of the delivery device 650 and/or may act as a manifold for fluidically coupling one or more inflation lumens 662A, 662B to the inflatable space-making element 652, such as via one or more internal inflation conduits 662C, 662D. The distal end cap 654 may house various lumens, such as one or more lumens for suction and/or one or more lumens visualization devices. In the illustrated embodiment, the inflatable space-making element 652 may be constructed generally in the form of a double-walled balloon, which may be molded as a single piece in a folded-over manner to create an internal chamber.

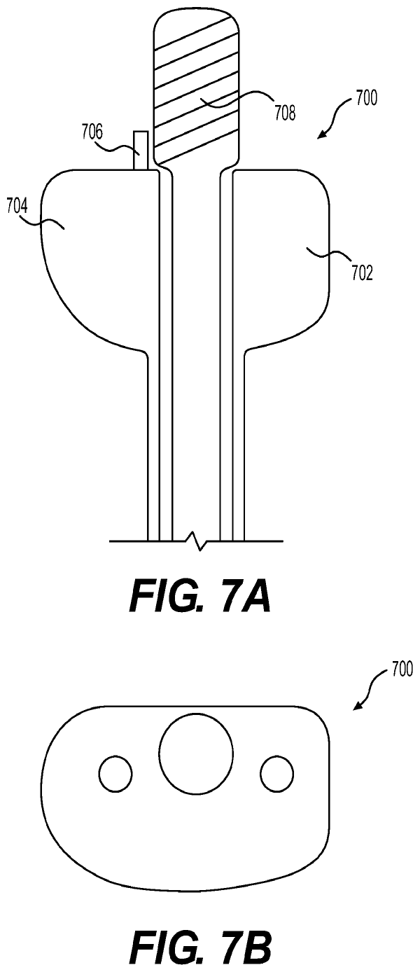

FIG. 7A is a simplified cross-sectional view of a distal portion of an example delivery device 700 and FIG. 7B is a distal view of the delivery device 700, all according to at least some aspects of the present disclosure. Delivery device 700 may be generally distinct from delivery devices 600, 650 in that delivery device 700 may create space proximal to the scope whereas delivery devices 600, 650 may create space 20 in a hollow-cored fashion such that the scope would reside within the boundaries of the inflatable space-making element 602, 652. Specifically, in the illustrated embodiment, the delivery device includes one or more expandable space-making elements 702, 704, which are disposed proximally from where the scope 706 emerges. In this embodiment, a therapeutic device 708 emerges laterally adjacent to the scope 706.

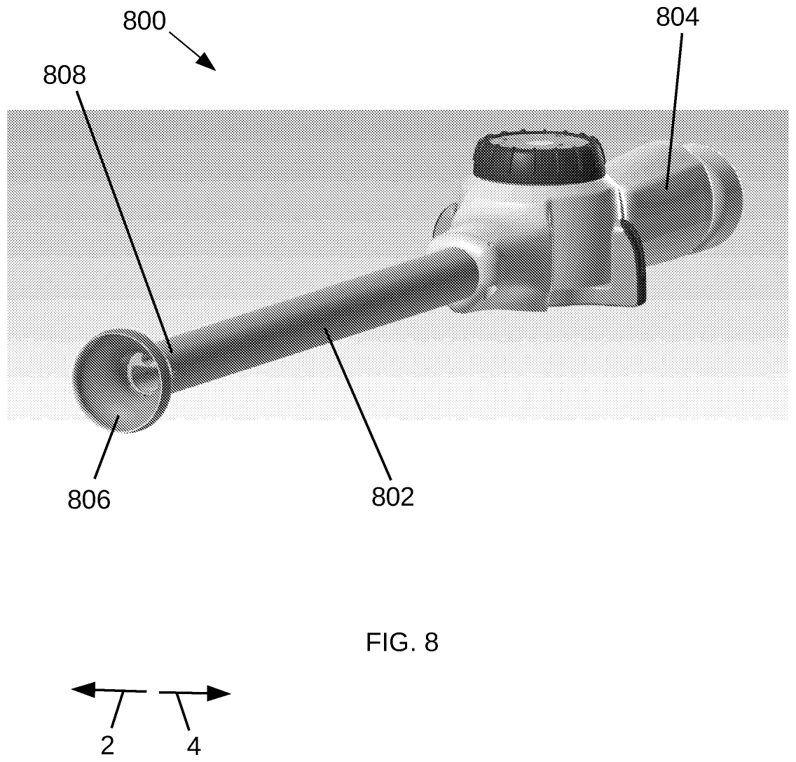

FIG. 8 is a distal perspective view of an example delivery device 800, according to at least some aspects of the present disclosure. Delivery device 800 may include a shaft 802, a handle 804, and/or a space-making element 806. The space-making element 806 may be disposed generally distally on the shaft 802, such as on the distal end portion 808 of the shaft 802.

In some example embodiments, the space-making element 806 may comprise a selectively deployable skirt movable between a retracted configuration, an intermediate configuration, and/or an extended configuration. For example, a skirt similar to that described in U.S. Patent Application Publication No. US2021/0008338, which is hereby incorporated by reference in its entirety, may be utilized in some example embodiments according to at least some aspects of the present disclosure. In some example embodiments according to at least some aspects of the present disclosure, the delivery device 800 may be configured for space creation on the posterior aspect of a patient's heart.

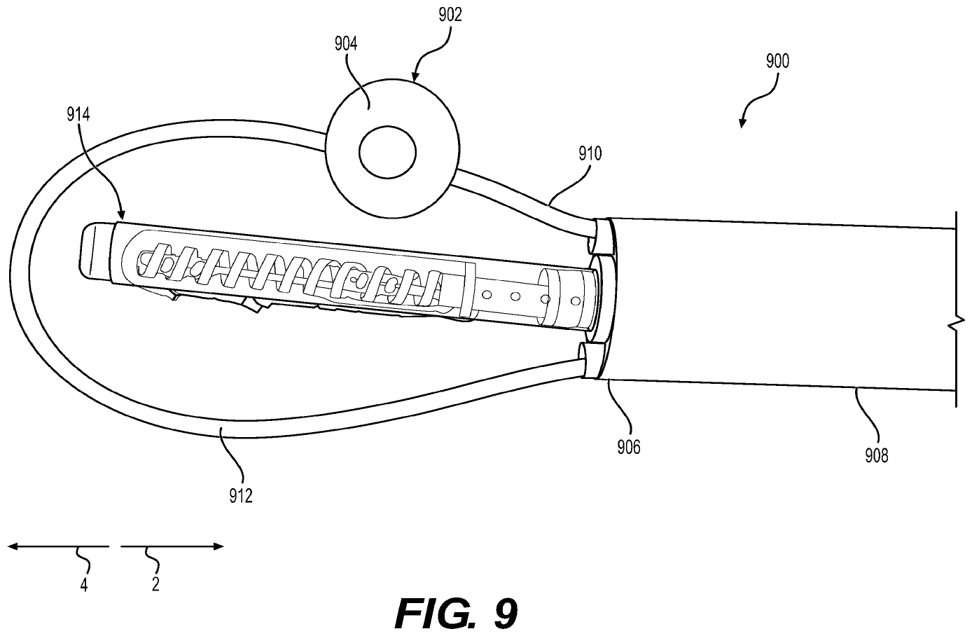

FIG. 9 is a perspective view of a distal portion of an example delivery device 900 comprising a movable space-making element 902, all according to at least some aspects of the present disclosure. In some example embodiments, a space-making element 902 may comprise an expandable element 904. The expandable element 904 may be movable with respect to a distal end portion 906 of the shaft 908 of the delivery device 900. For example, the expandable element 904 may be disposed on a guidewire 910, which may be routed through a lumen of the delivery device 900. The guidewire 910 may be extended and/or retracted to position the expandable element 904 as desired. In some example embodiments, the expandable element 904 may comprise an inflatable element, such as a balloon. In some example embodiments, the inflatable element may be operatively connected to a source of inflation fluid via a lumen 912. In some example embodiments, a therapeutic element 914 may be delivered via the delivery device 900.

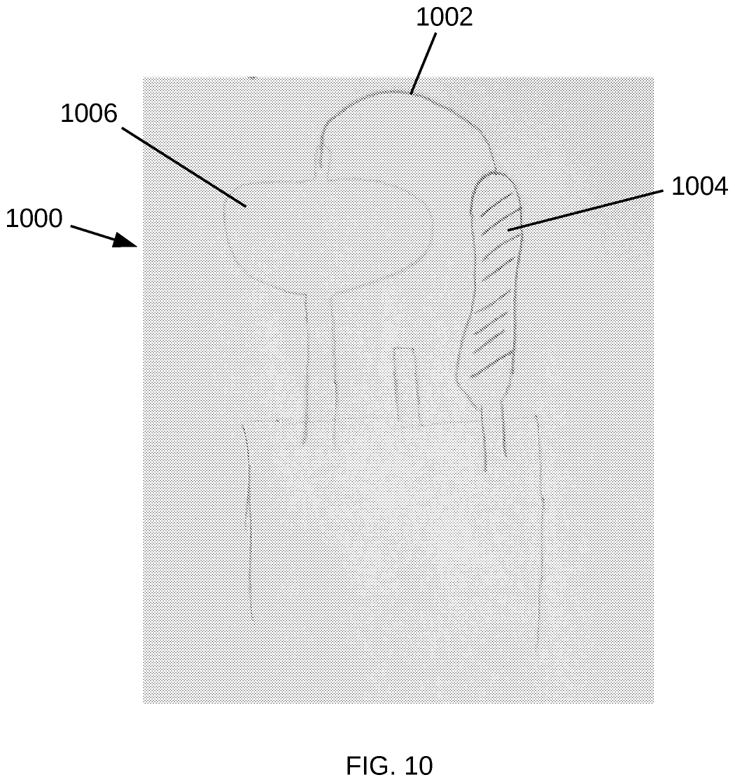

FIG. 10 is a simplified side view of a distal portion of an example delivery device 1000, according to at least some aspects of the present disclosure. This example embodiment is generally similar to the embodiment illustrated in FIG. 9. In this example embodiment, a guidewire 1002 may extend between the therapeutic element 1004 and the movable expandable element 1006.

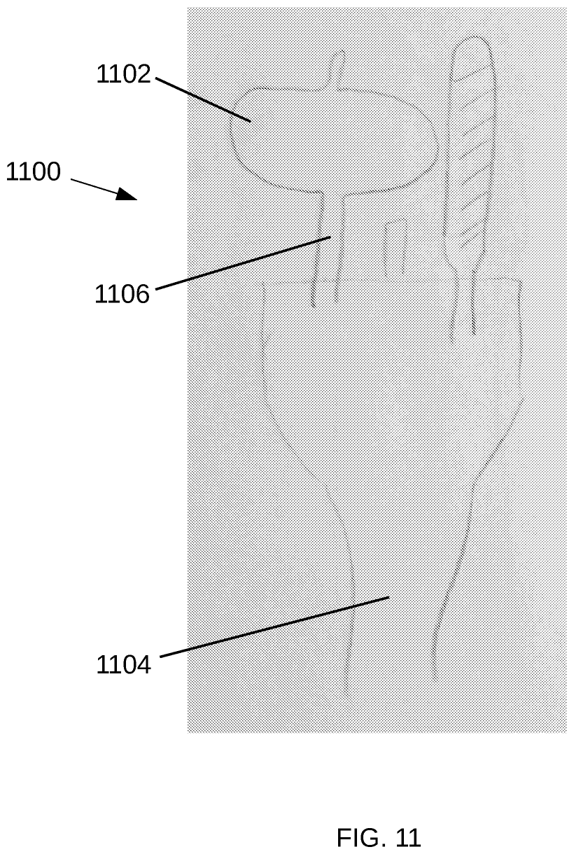

FIG. 11 is a simplified side view of a distal portion of an example delivery device 1100, according to at least some aspects of the present disclosure. This example embodiment is generally similar to the embodiments illustrated in FIGS. 9 and 10. In this example embodiment, a guidewire may not be used. Accordingly, in some example embodiments, the movable expandable element 1102 may be coupled to the shaft 1104 only via the lumen 1106.

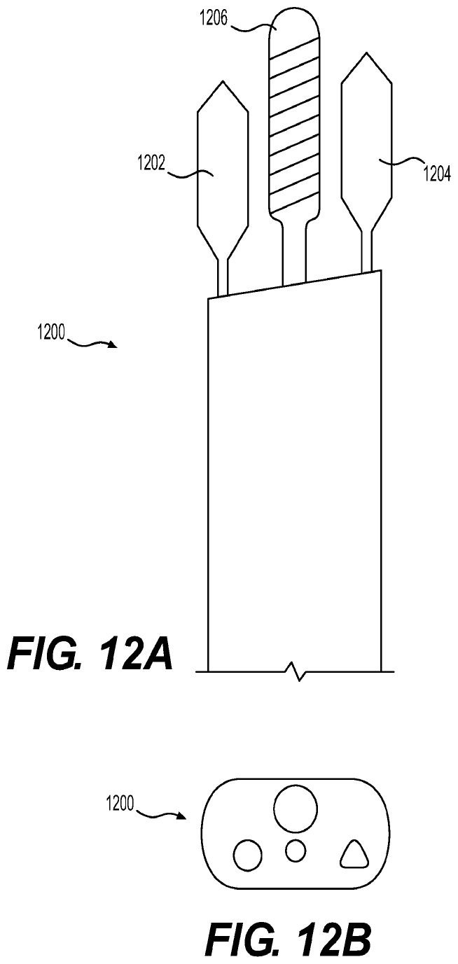

FIG. 12A is a simplified side view of a distal portion of an example delivery device 1200 and FIG. 12B is a distal view of the delivery device 1200, all according to at least some aspects of the present disclosure. This example embodiment may be generally similar to the embodiment illustrated in FIG. 11. Some example embodiments may comprise two or more expandable elements, such as inflatable expandable elements 1202, 1204. In some example embodiments, a therapeutic device 1206 may be positioned generally between the inflatable expandable elements 1202, 1204.

In some alternative example embodiments, one or more expandable elements, such as an inflatable expandable element, may be disposed on a stylet instead of or in addition to a guidewire. In some alternative embodiments, a mechanically expandable element may be utilized instead of or in addition to an inflatable expandable element. More generally, inflatable and/or mechanically expandable elements may be disposed on guidewires and/or stylets in various example embodiments according to at least some aspects of the present disclosure.

As mentioned above with reference to FIG. 1, various example delivery devices 100 according to at least some aspects of the present disclosure may include one or more lumens 106, 108 configured to receive one or more visualization devices 14 therethrough. In some example embodiments, one or more of the lumens 106, 108 may be specifically dedicated for use with one or more visualization devices 14, such as endoscopes.

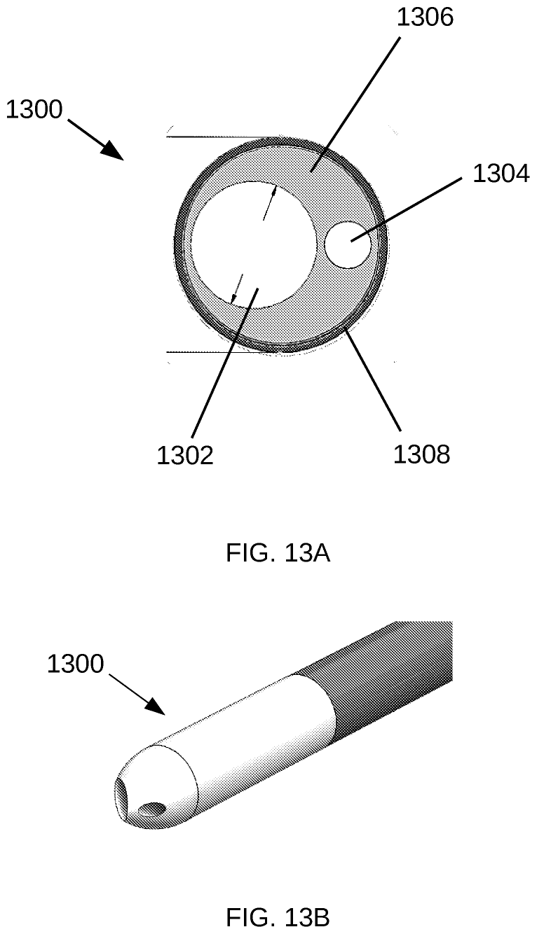

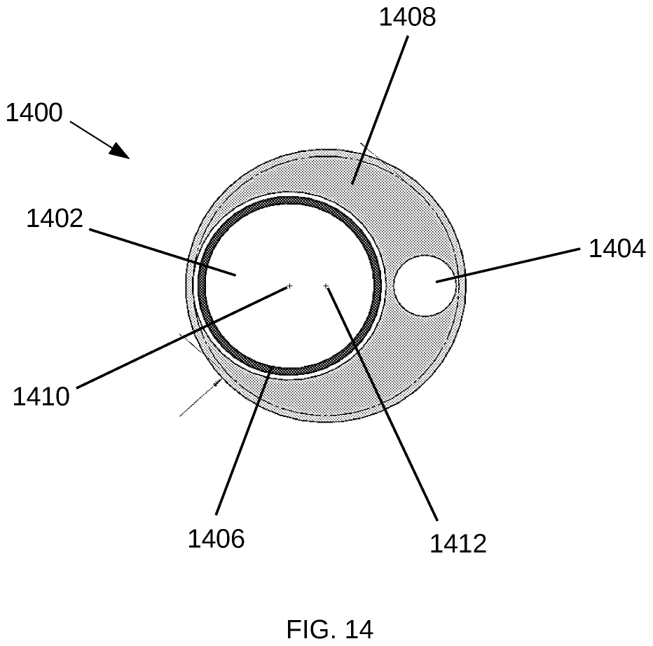

Some example embodiments may be configured to rotationally reposition one or more lumens 106, 108 relative to the handle 102. FIG. 13A is a simplified cross-sectional view of an example shaft 1300, according to at least some aspects of the present disclosure. In this example embodiment, the shaft 1300 may comprise a therapeutic device lumen 1302 and/or a visualization device lumen 1304, as well as other lumens as described herein. The lumens 1302, 1304 may be disposed in an inner shaft portion 1306. The inner shaft portion 1306 may be disposed concentrically or eccentrically within an outer shaft portion 1308. In some example embodiments, the inner shaft element 1306 may be rotatable generally about the longitudinal axis of the shaft 1300. In some example embodiments, the outer shaft element 1308 may be steerable in one or more directions. FIG. 13B is a perspective view of the distal end portion of the shaft 1300 of FIG. 13A.

FIG. 14 is a simplified cross-sectional view of an example shaft 1400, according to at least some aspects of the present disclosure. In this example embodiment, the shaft 1400 may comprise a therapeutic device lumen 1402 and/or a visualization device lumen 1404. The shaft 1400 may comprise an inner shaft portion 1406 and/or an outer shaft portion 1408. In some example embodiments, the inner shaft portion 1406 may be steerable. In some example embodiments, the outer shaft portion 1408 may have a generally circular circumferential perimeter. The therapeutic device lumen 1402 may be disposed in the inner shaft portion 1406. The visualization device lumen may be disposed in the outer shaft portion 1408. The outer shaft portion may be rotatable about the inner shaft portion 1406. In some example embodiments, the inner shaft portion 1406 may disposed non-concentrically with the outer shaft portion 1408. In some example embodiments, the central axis 1410 of the inner shaft portion 1406 may be laterally offset from the central axis 1412 of the outer shaft portion 1408. In some example embodiments, the outer shaft portion 1408 may rotate eccentrically relative to the inner shaft portion 1406.

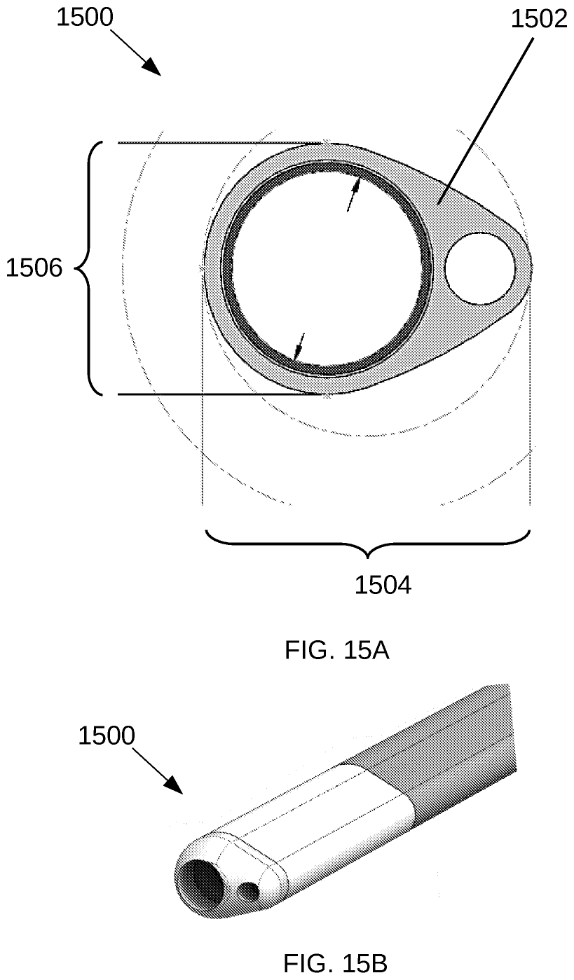

FIG. 15A is a simplified cross-sectional view of an example shaft 1500, according to at least some aspects of the present disclosure. Example shaft 1500 may be generally similar to the embodiment described above with reference to FIG. 14. In some example embodiments, the shaft 1500 may comprise an outer shaft portion 1502 having a non-circular outer perimeter. In some example embodiments, the shaft 1500 may have a maximum outer width 1504 in one direction that is substantially greater than the maximum outer width 1506 in a perpendicular direction. FIG. 15B is a perspective view of the distal end portion of the shaft 1500 of FIG. 15A.

Some example embodiments may include one or more lumens that may be utilized for purposes other than receiving visualization devices and/or therapeutic devices. For example, some embodiments may include one or more separate lumens configured for infusion and/or aspiration (e.g., suction) of fluids during a procedure.

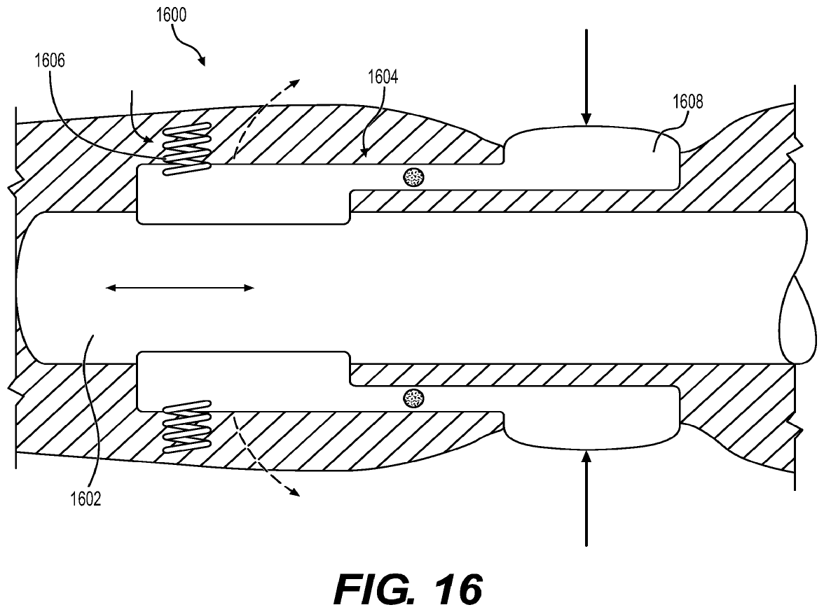

Some example embodiments according to at least some aspects of the present disclosure may include features configured to synchronize movement of therapeutic and/or visualization devices. For example, a feature for synchronizing an endoscope and a delivery device (e.g., cannula) may comprise a Tuohy-Borst style fitting or a lock-brake mechanism that temporarily or constantly grips a section of an endoscope shaft until released by a handle feature. In other example embodiments, such a feature may include a cradle-receiver for the scope head. FIG. 16 is a simplified cross-sectional view of a delivery device 1600 and an endoscope 1602, according to at least some aspects of the present disclosure. The delivery device 1600 may comprise one or more active lock-brake mechanisms 1604. In some example embodiments, a lock-brake mechanism 1604 may comprise a spring 1606 biased, pivotably disposed arm 1608. One end of the arm 1608 may be spring-biased to releasably engage the endoscope 1602. The opposite end of the arm 1608 may be accessible to a user. When the opposite end of the arm 1608 is depressed, for example, the arm 1608 may pivot out to disengage from the endoscope 1602. In alternative embodiments, a passive lock may be utilized to releasably couple the delivery device 1600 and the endoscope 1602.

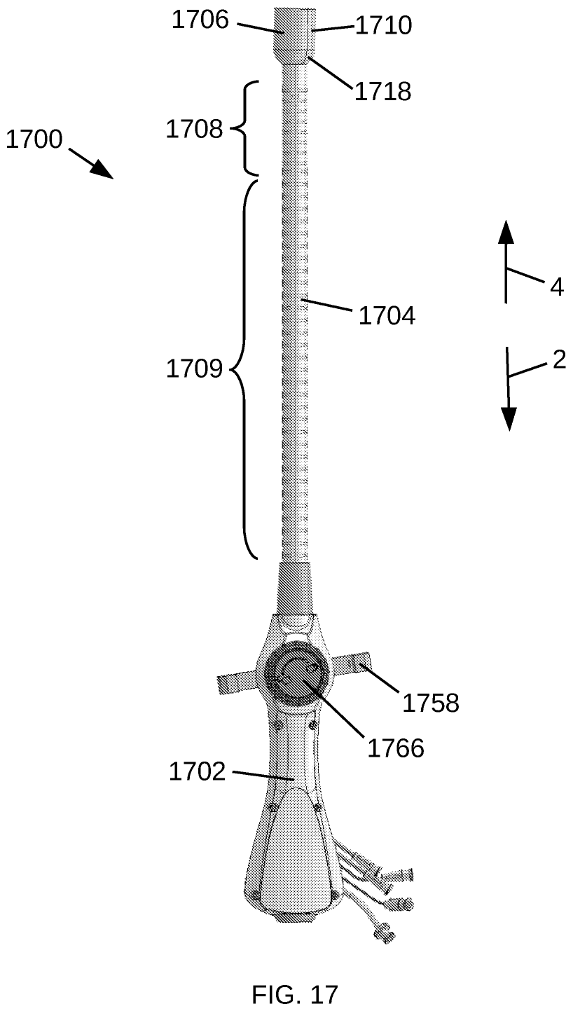

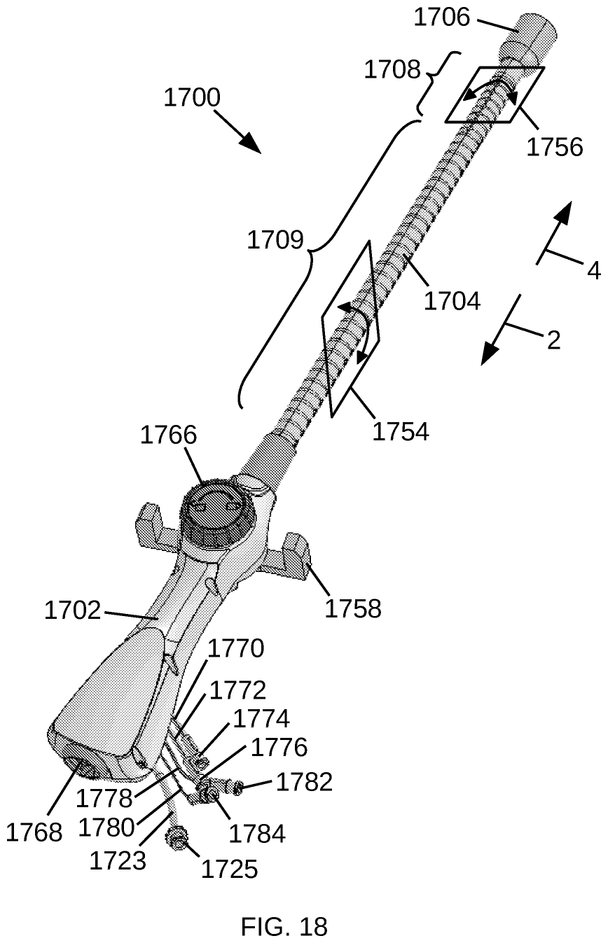

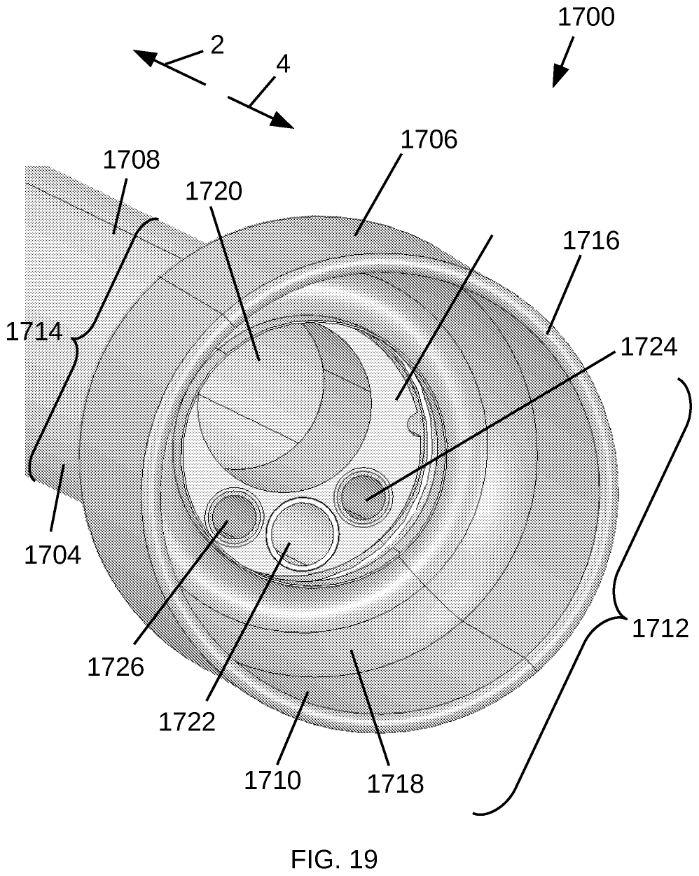

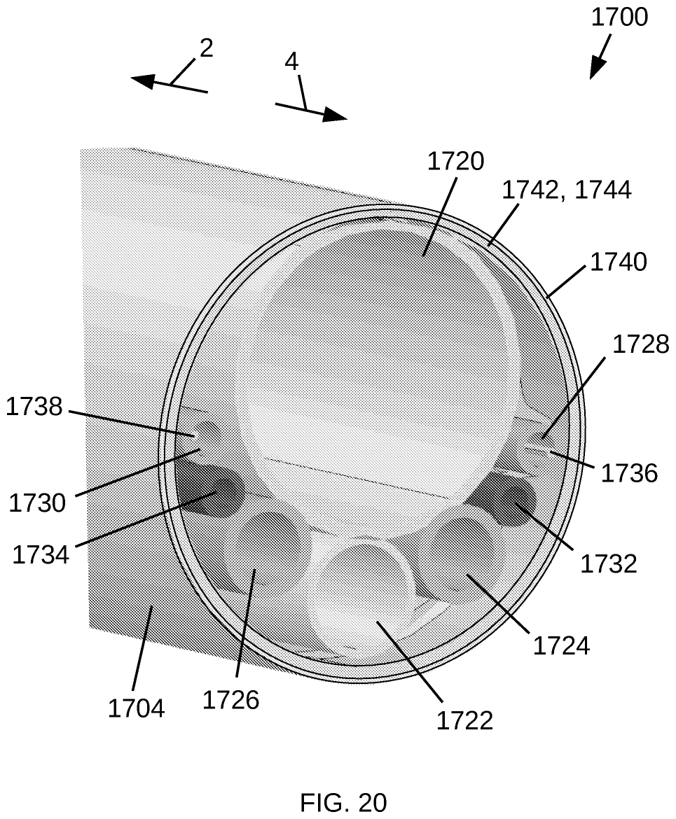

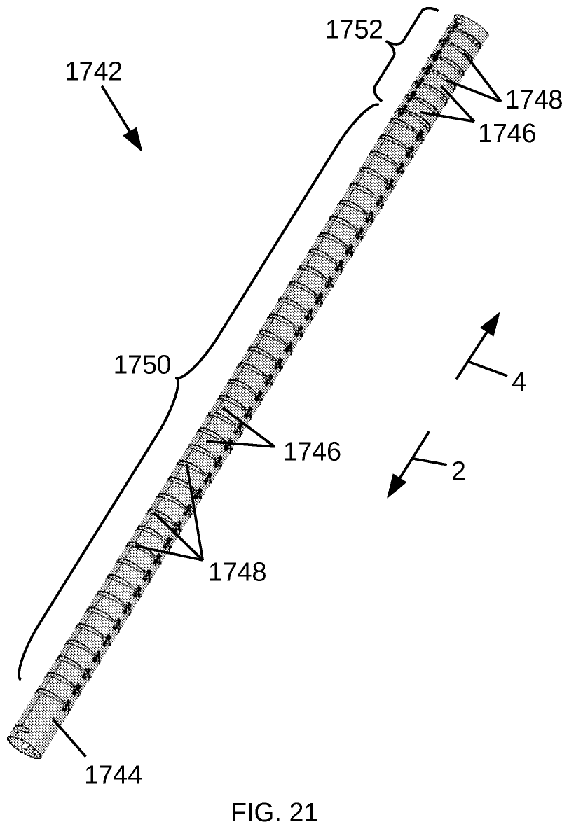

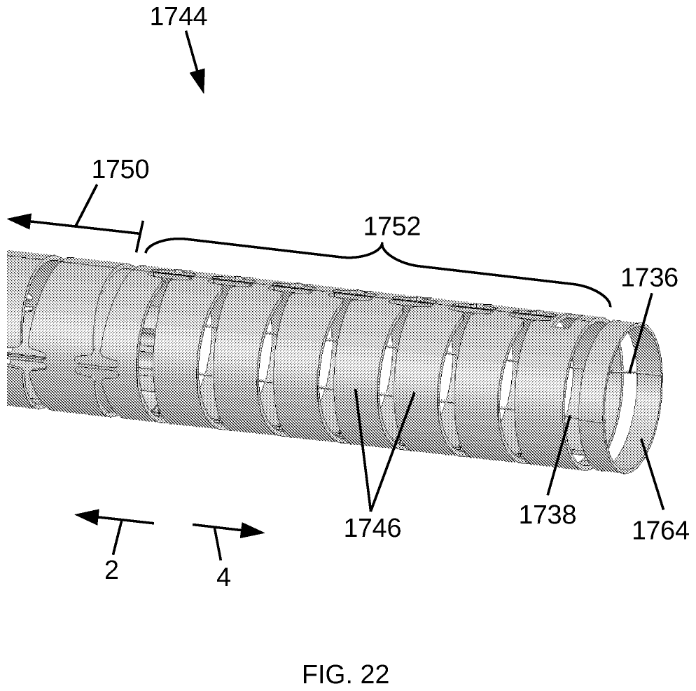

FIG. 17 is a top view of an example delivery device 1700, FIG. 18 is a proximal perspective view of the delivery device 1700 of FIG. 17, FIG. 19 is a detailed distal perspective view of the delivery device 1700 of FIG. 17, FIG. 20 is a detailed distal perspective cross-section view of the shaft of the delivery device 1700 of FIG. 17, FIG. 21 is a proximal perspective view of a structural component of the shaft of the delivery device 1700 of FIG. 17, and FIG. 22 is a detailed perspective view of a distal end portion of the structural component of the shaft of the delivery device 1700 of FIG. 17, all according to at least some aspects of the present disclosure. Generally, it will be understood that the delivery device 1700 is an example combination of various features described throughout the present disclosure and that any alternative features described elsewhere herein may utilized in generally similar alternative embodiments.

Referring to FIGS. 17 and 18, in the illustrated embodiment, the delivery device 1700 may include a proximally disposed handle 1702, an elongated shaft 1704 extending distally from the handle 1702, and/or a space-making element 1706 disposed distally on a distal end portion 1708 of the shaft 1704. The distal end portion 1708 may be disposed distally relative to a proximal portion 1709 of the shaft 1704.

Referring to FIGS. 17-19, in the illustrated embodiment, the space-making element 1706 may comprise a reconfigurable space-making element, such as an inflatable space-making element. In FIGS. 17-19, the inflatable space-making element is shown in an inflated (e.g., deployed and/or expanded) configuration. The inflatable space-making element 1706 may be reconfigured to a deflated (e.g., retracted) configuration as described elsewhere herein. In some example embodiments, at least a portion 1710 of the inflatable space-making element 1706 may be generally in the form of a right circular cylinder, which may be generally coaxially disposed with the shaft 1704. The inflatable space-making element 1706 may be inflatable to have an inflated diameter 1712 that is substantially greater than a diameter 1714 of the shaft 1704. In the illustrated embodiment, the inflatable space-making element 1706 includes a distal end face 1716 that is generally perpendicular to the cylindrical portion 1710 of the inflatable space-making element 1706. The inflatable space-making element 1706 may include a generally conically tapered transition portion 1718 interposing the cylindrical portion 1710 and the shaft 1704. In the illustrated embodiment, the inflatable space-making element 1706 may be constructed with a single internal inflatable chamber. In other example embodiments, the inflatable space-making element 1706 may be constructed with two or more fluidically separated internal inflatable chambers.

Referring to FIGS. 19 and 20, in the illustrated embodiment, the shaft 1704 may include a plurality of longitudinally oriented lumens, such as one or more therapeutic device lumens 1720, one or more visualization device lumens 1722, one or more suction lumens 1724, 1726, one or more steering lumens 1728, 1730, and/or one or more inflation lumens 1732, 1734.

In the illustrated embodiment, a steering wire 1736, 1738 is movably disposed within each steering lumen 1728, 1730. In other embodiments, steering wires 1736, 1738 may be routed through the shaft 1704 without steering lumens 1728, 1730 and/or mechanical connectors other than wires (e.g., rods, cables, etc.) may be utilized in place of the steering wires 1736, 1738.

In some example embodiments, one or more of the suction lumens 1724, 1726 may be used to convey fluid to the surgical site, such as for irrigation. Similarly, in some example embodiments, one or more of the inflation lumens 1732, 1734 may be used to remove inflation fluid from the inflatable space-making element 1706.

In the illustrated embodiment, various lumens 1720, 1722, 1724, 1726, 1728, 1730, 1732, 1734 may be aligned generally straight and generally in parallel along the longitudinal axis of the shaft 1704. In alternative embodiments, one or more of the lumens 1720, 1722, 1724, 1726, 1728, 1730, 1732, 1734 may be disposed in a non-straight and/or non-parallel orientation.

Referring to FIGS. 20-22, in the illustrated embodiment, the shaft 1704 may include a generally tubular outer jacket 1740, which may be formed from a thermoplastic elastomer, such as polyether block amide (e.g., PEBA). The shaft 1704 may include a structural component 1742 disposed at least partially radially within the jacket 1740. In the illustrated embodiment, the structural component 1742 may include a laser cut, stainless steel hypotube 1744.

Referring to FIGS. 21-22, the hypotube 1744 may be generally in the form of an elongated, tubular, right circular cylinder. The hypotube 1744 may include a plurality of solid portions 1746 and/or a plurality of gaps 1748. In some example embodiments, the solid portions 1746 and the gaps 1748 may be arranged in a generally alternating pattern along the length of the hypotube 1744. In some example embodiments, the size, shape, arrangement, and/or orientation of the solid portions 1746 and/or the gaps 1748 (or other characteristics of the hypotube 1744) may be different in different portions 1750, 1752 of the hypotube 1744 and/or may vary gradually over the length of the hypotube 1744.

In the illustrated embodiment, the proximal portion 1750 of the hypotube 1744, which may correspond to the proximal portion 1709 of the shaft 1704 (FIGS. 17-18), may be configured to allow up-down bending, while preventing substantial left-right bending. In the illustrated embodiment, the left-right plane may be substantially perpendicular to the up-down plane. Specifically, in the illustrated embodiment, the upper and lower portions of the proximal portion 1750 may include laterally oriented, generally circumferentially arranged gaps 1748 and/or the left and right portions of the proximal portion 1750 may be substantially solid. In the illustrated embodiment, the upper and lower portions of the proximal portion 1750 may be generally symmetrical, thus allowing symmetric, bi-directional bending. In some alternative embodiments, the upper and lower portions of the proximal portion 1750 may differ to provide uni-directional bending and/or asymmetric bending.

In the illustrated embodiment, the distal portion 1752 of the hypotube 1744, which may correspond to the distal portion 1708 of the shaft 1704, (FIGS. 17-18) may be configured to allow left-right bending, while preventing substantial up-down bending. Specifically, in the illustrated embodiment, the left and right portions of the distal portion 1752 may include laterally oriented, generally circumferentially arranged gaps 1748 and/or the upper and lower portions of the distal portion 1752 may be substantially solid. In the illustrated embodiment, the upper and lower portions of the distal portion 1752 may be generally symmetrical, thus allowing symmetric, bi-directional bending. In some alternative embodiments, the upper and lower portions of the distal portion 1752 may differ to provide uni-directional bending and/or asymmetric bending.

In some alternative embodiments, the hypotube 1744 may include more than two portions (e.g., proximal portion 1750 and distal portion 1752). In some alternative embodiments, the hypotube 1744 may be configured for bending in non-perpendicular planes. In some alternative embodiments, different portions 1750, 1752 of the hypotube 1744 may be configured for bending in substantially the same plane, such as to different extents and/or in generally opposite directions.

Referring to FIGS. 17, 18, and 22, in the illustrated embodiment, the proximal portion 1709 of the shaft 1704 of the delivery device 1700 may be generally flexible up-down, such as in a generally vertical plane 1754. In the illustrated embodiment, the distal portion 1708 of the shaft 1704 may be steerable left-right, such as in a generally horizontal plane 1756, by operation of an actuator 1758 disposed on the handle 1702. The actuator 1758 may be operatively connected to steer the distal portion 1708 of the shaft 1704 by one or more mechanical connectors, such as steering (pull) wires 1736, 1738. The pull wires 1736, 1738 may be coupled to opposite (e.g., left and right) aspects of a distally positioned pull ring 1764. In operation, the actuator 1758 may be rotated to pull on one of the pull wires 1736, 1738, which may cause the corresponding portion of the pull ring 1764 to move proximally, thus causing the distal portion 1708 of the shaft 1704 to steer generally towards the pulled pull wire 1736, 1738.

Referring to FIGS. 17 and 18, the handle 1702 may include a locking actuator 1766. The locking actuator 1766 may be operable (e.g., rotatable) to lock and unlock the steering actuator 1758. Accordingly, the distal portion 1708 of the shaft 1704 may be maintained in a partially or fully steered position.

In some alternative example embodiments, the distal portion 1708 of the shaft 1744 may be generally flexible up-down and/or left-right when the pull wires 1736, 1738 are not tensioned (e.g., by the actuator 1758). In use, such an embodiment may be steered (e.g., by the actuator 1758) during some portions of a procedure and/or may be flexed (without active steering) during other portions of a procedure. For example, steering may be used to actively direct the distal portion 1708 of the shaft 1704 while the delivery device 1700 is routed to the posterior side of the patient's heart. Then, the pull wires 1736, 1738 may be slacked and the distal portion 1708 of the shaft 1704 may be repositioned in a passive flexing configuration as the delivery device 1700 is positioned at a surgical site near the superior portion of the patient's heart. In some embodiments, adjacent patient anatomy may constrain movement of the distal portion 1708 in one or more directions.

Referring to FIGS. 18 and 20, the delivery device 1700 may include one or more interface elements associated with one or more components of the delivery device 1700. For example, the therapeutic device lumen 1720 and/or the visualization device lumen 1722 may be operatively connected to a proximal port 1768, which may be disposed proximally on the handle 1702. The proximal port 1768 may allow insertion of a therapeutic device and/or a visualization device into their respective lumens 1720, 1722. In the illustrated embodiment, the therapeutic device lumen 1720 and the visualization device lumen 1722 may come together in a wye within the handle 1702. Thus, the proximal port 1768 provides access to both the therapeutic device lumen 1720 and the visualization device lumen 1722. In some example embodiments, the proximal port 1768 may comprise a hemostasis valve configured to limit flow of fluids through the proximal port 1768 to and/or from the therapeutic device lumen 1720 and/or the visualization device lumen 1722. In alternative embodiments, each of the therapeutic device lumen 1720 and the visualization device lumen 1722 may be provided with a separate proximal opening. In the illustrated embodiment, the therapeutic device lumen 1720 may be fluidically connected to a proximal fluid conduit 1723 and/or a fluid connector 1725.

In the illustrated embodiment, the suction lumens 1724, 1726 may be fluidically connected to respective proximal suction conduits 1770, 1772 and/or respective suction connectors 1774, 1776. In some alternative embodiments, more than one suction lumen 1724, 1726 may be fluidically coupled to one proximal suction conduit 1770, 1772 and/or suction connector 1774, 1776. Similarly, in some alternative embodiments, more than one suction conduit 1770, 1772 and/or suction connector 1774, 1776 may be fluidically coupled to one suction lumen 1724, 1726.

In the illustrated embodiment, the inflation lumens 1732, 1734 may be fluidically connected to respective proximal inflation conduits 1778, 1780 and/or respective inflation connectors 1782, 1784. In some alternative embodiments, more than one inflation lumen 1732, 1734 may be fluidically coupled to one proximal inflation conduit 1778, 1780 and/or inflation connector 1782, 1784. Similarly, in some alternative embodiments, more than one inflation conduit 1778, 1780 and/or inflation connector 1782, 1784 may be fluidically coupled to one inflation lumen 1732, 1734.

Some example embodiments may include lumens for additional purposes, such as accessory lumens for additionally, accessory surgical tools. Some such accessory lumens may be generally similar in structure to the suction lumens 1724, 1726 illustrated in FIG. 20; however, the proximal ends may be configured to receive an accessory surgical tool therethrough, similar to the therapeutic device lumen 1720 and/or the visualization device lumen 1722. Similarly, in some example embodiments utilizing a chip-on-tip visualization device configuration, the lumen 1722 described above as a visualization lumen may be utilized as an accessory lumen.

In some example embodiments, inflatable elements described herein may be constructed of polyether block amide (e.g., PEBA), polyvinyl chloride (e.g., PVC), polyethylene terephthalate (e.g., PET), and/or urethane, for example.

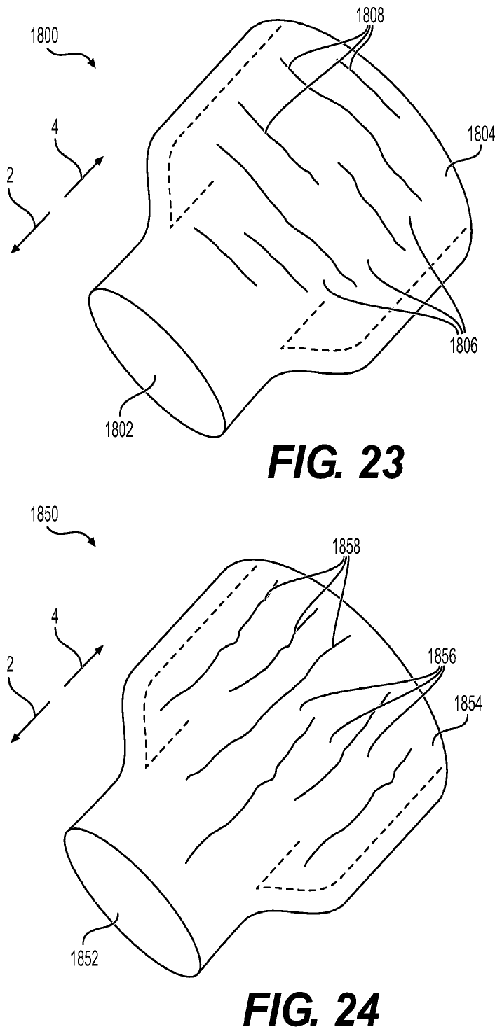

FIG. 23 is a proximal perspective view of an alternative example inflatable space-making element 1800 including circumferential ribs, according to at least some aspects of the present disclosure. The inflatable space-making element 1800 may be utilized in place of other space-making elements described herein, such as the space-making element 1706 of the embodiment of FIG. 17. The inflatable space-making element 1800 may include a proximal portion 1802 configured to operatively couple to a delivery device shaft and a distally disposed expandable portion 1804. The expandable portion 1804 may be configured with a plurality of generally circumferentially oriented ribs 1806. The ribs 1806 may be formed by generally circumferentially disposed secondary welds 1808 connecting radially inner and outer walls of the expandable portion 1804.

FIG. 24 is a proximal perspective view of an alternative example inflatable space-making element 1850 including longitudinal ribs, according to at least some aspects of the present disclosure. The inflatable space-making element 1850 may be utilized in place of other space-making elements described herein, such as the space-making element 1706 of the embodiment of FIG. 17. The inflatable space-making element 1850 may include a proximal portion 1852 configured to operatively couple to a delivery device shaft and a distally disposed expandable portion 1854. The expandable portion 1854 may be configured with a plurality of generally longitudinally oriented ribs 1856. The ribs 1856 may be formed by generally longitudinally disposed secondary welds 1858 connecting radially inner and outer walls of the expandable portion 1854.

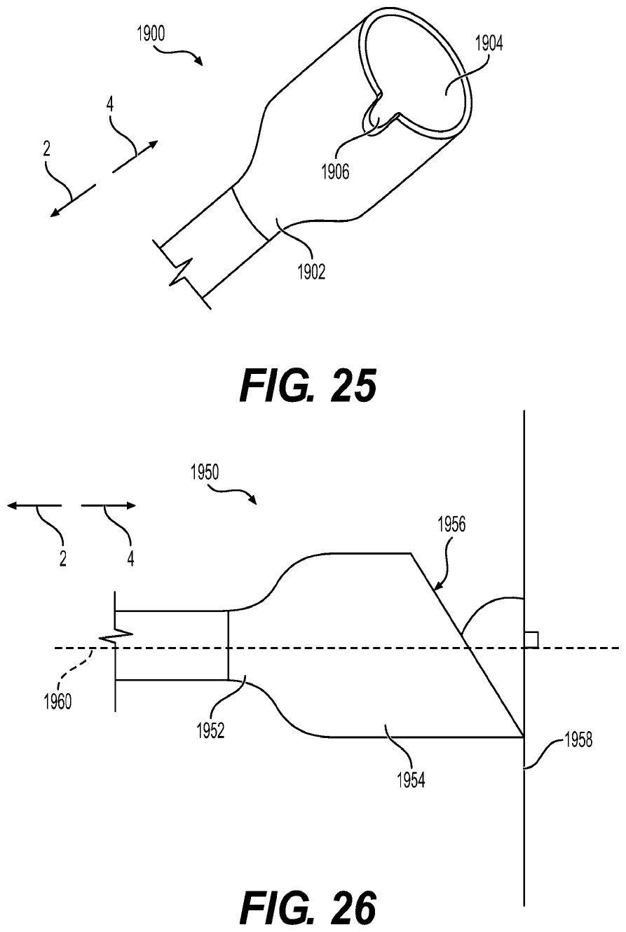

FIG. 25 is a distal perspective view of an alternative example inflatable space-making element 1900 including an orientation feature, according to at least some aspects of the present disclosure. The inflatable space-making element 1900 may be utilized in place of other space-making elements described herein, such as the space-making element 1706 of the embodiment of FIG. 17. The inflatable space-making element 1900 may include a proximal portion 1902 configured to operatively couple to a delivery device shaft and a distally disposed expandable portion 1904. The expandable portion 1904 may be include an orientation feature, such as a notch 1906. The orientation feature, such as the notch 1906, may facilitate determination of the rotational orientation of the delivery device, such as when the distal portion including the expandable portion is observed through a visualization device (e.g., an endoscope).

FIG. 26 is a side view of an alternative example inflatable space-making element 1950 including an angled distal face, according to at least some aspects of the present disclosure. The inflatable space-making element 1950 may be utilized in place of other space-making elements described herein, such as the space-making element 1706 of the embodiment of FIG. 17. The inflatable space-making element 1950 may include a proximal portion 1952 configured to operatively couple to a delivery device shaft and a distally disposed expandable portion 1954. The expandable portion 1954 may be include a distal face 1956, which may be angled relative to a plane 1958 normal to a longitudinal axis 1960 of a shaft of the delivery device. The angled distal face 1956 may facilitate separation of biological tissues, such as separating the pericardium from the epicardium.

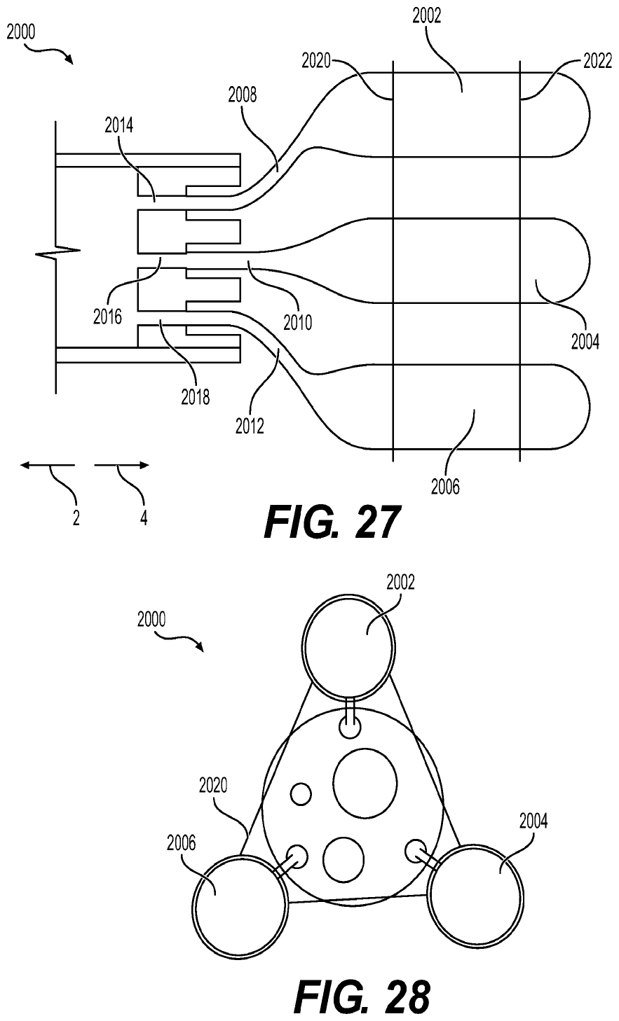

FIG. 27 is a simplified side cross-section view of an alternative inflatable space-making element 2000 including a plurality of longitudinally orientated inflatable elements and FIG. 28 is a simplified distal end view of the inflatable space-making element 2000 of FIG. 27, all according to at least some aspects of the present disclosure. The inflatable space-making element 2000 may be utilized in place of other space-making elements described herein, such as the space-making element 1706 of the embodiment of FIG. 17. In the illustrated embodiment, the inflatable space-making element 2000 may include a plurality (e.g., three) of elongated, longitudinally oriented inflatable elements 2002, 2004, 2006. In this embodiment, each inflatable element 2002, 2004, 2006 may be generally in the form of an elongated, expanded, generally circular tube having a closed distal end and/or a substantially unexpanded proximal portion acting as a conduit 2008, 2010, 2012 between the expanded portion and a proximal end fluidically coupled to one or more inflation lumens 2014, 2016, 2018. The inflatable space-making element 2000 may include one or more collapsible structural elements 2020, 2022, such as nitinol wires, configured to facilitate a desired spacing and/or arrangement of the inflatable elements 2002, 2004, 2006. For example, the structural elements 2020, 2022 may form a collapsible nitinol frame.

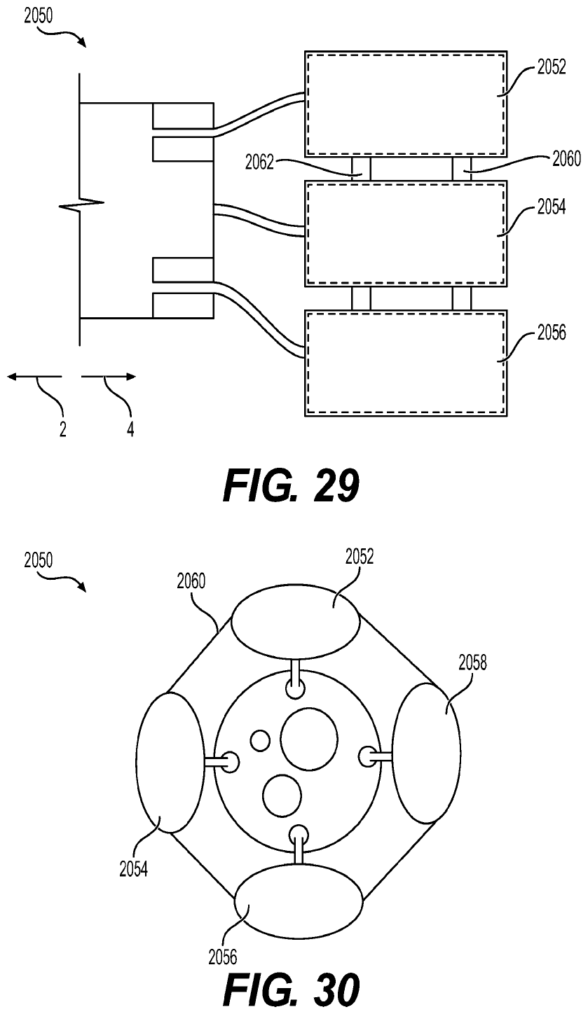

FIG. 29 is a simplified side cross-section view of an alternative inflatable space-making element 2050 including a plurality of flattened longitudinally orientated inflatable elements and FIG. 30 is a simplified distal end view of the inflatable space-making element 2050 of FIG. 29, all according to at least some aspects of the present disclosure. The inflatable space-making element 2050 may be utilized in place of other space-making elements described herein, such as the space-making element 1706 of the embodiment of FIG. 17. In the illustrated embodiment, the inflatable space-making element 2050 may include a plurality (e.g., four) of elongated, flattened, longitudinally oriented inflatable elements 2052, 2054, 2056, 2058. In this embodiment, each inflatable element 2052, 2054, 2056, 2058 may be generally in the form of an elongated, expanded tube having a generally oval cross section. Each inflatable element 2052, 2054, 2056, 2058 may include a closed distal end and/or a substantially unexpanded proximal portion acting as a conduit between the expanded portion and a proximal end fluidically coupled to one or more inflation lumens. The inflatable space-making element 2050 may include one or more collapsible structural elements 2060, 2062, such as flexible tabs, configured to facilitate a desired spacing and/or arrangement of the inflatable elements 2052, 2054, 2056, 2058.

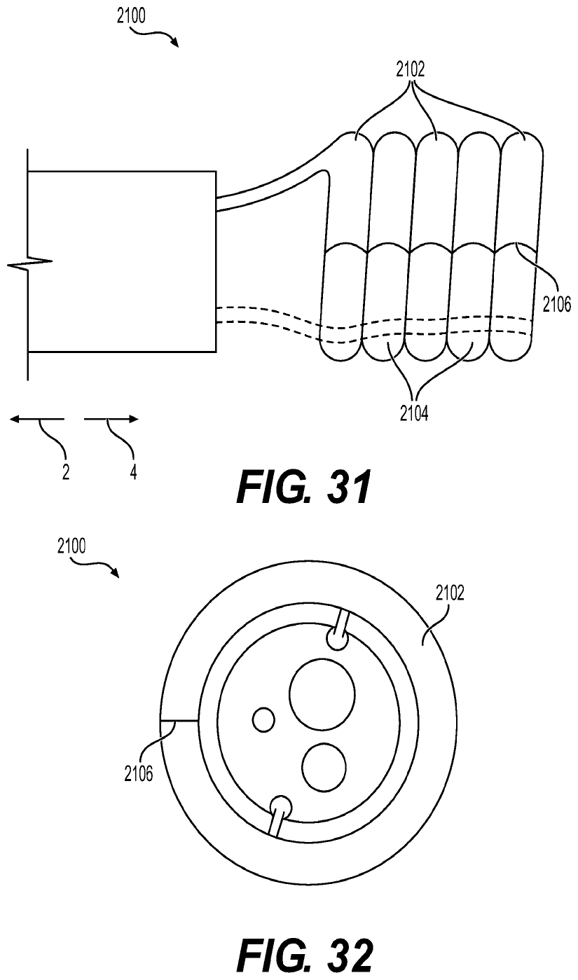

FIG. 31 is a simplified side view of an alternative inflatable space-making element 2100 including coiled inflatable elements and FIG. 32 is a simplified distal end view of the inflatable space-making element 2100 of FIG. 31, all according to at least some aspects of the present disclosure. The inflatable space-making element 2100 may be utilized in place of other space-making elements described herein, such as the space-making element 1706 of the embodiment of FIG. 17. In the illustrated embodiment, the inflatable space-making element 2100 may include one or more elongated, generally helically arranged inflatable elements 2102, 2104. In this embodiment, each inflatable element 2102, 2104 may be generally in the form of an elongated, expanded tube having a generally circular cross section. Each inflatable element 2102, 2104 may include a closed distal end and/or a substantially unexpanded proximal portion acting as a conduit between the expanded portion and a proximal end fluidically coupled to one or more inflation lumens. In the illustrated embodiment, the two generally helical inflatable elements 2101, 2104 may be disposed in an alternating arrangement. The inflatable space-making element 2100 may include one or more collapsible structural elements 2106, such as nitinol wire, configured to facilitate a desired spacing and/or arrangement of the inflatable elements 2102, 2104.

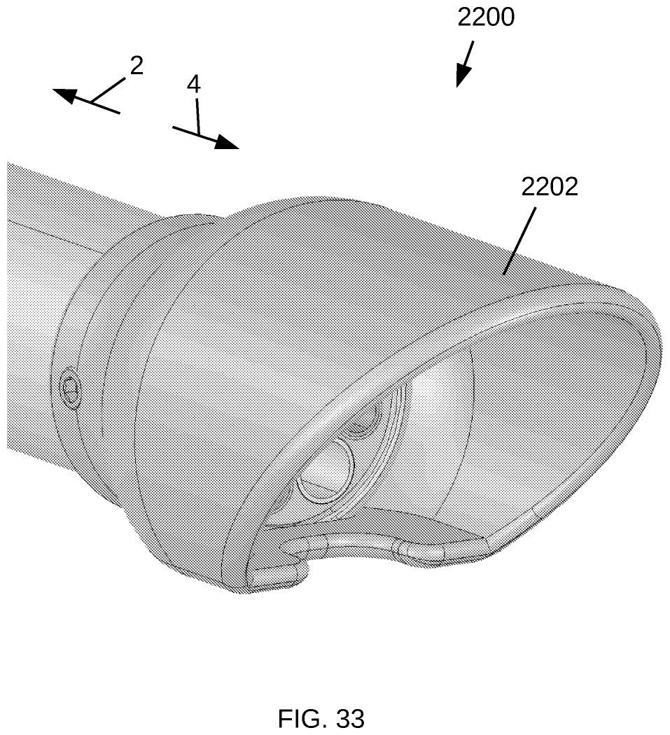

FIG. 33 is a distal perspective view of a distal end portion of an alternative delivery device 2200, in accordance with at least some aspects of the present disclosure. Generally, delivery device 2200 is similar to the delivery device 1700 of FIG. 17; however, delivery device 2200 includes a rigid space-making element 2202 instead of an inflatable space-making element 1706. In the illustrated embodiment, the rigid space-making element 2202 is generally similar to the rigid space-making elements 518, 530 illustrated in FIGS. 5D and 5G. Some example embodiments including only rigid space-making elements (e.g., without inflatable space-making elements) may not include inflation lumens as described herein in connection with embodiments including inflatable elements.

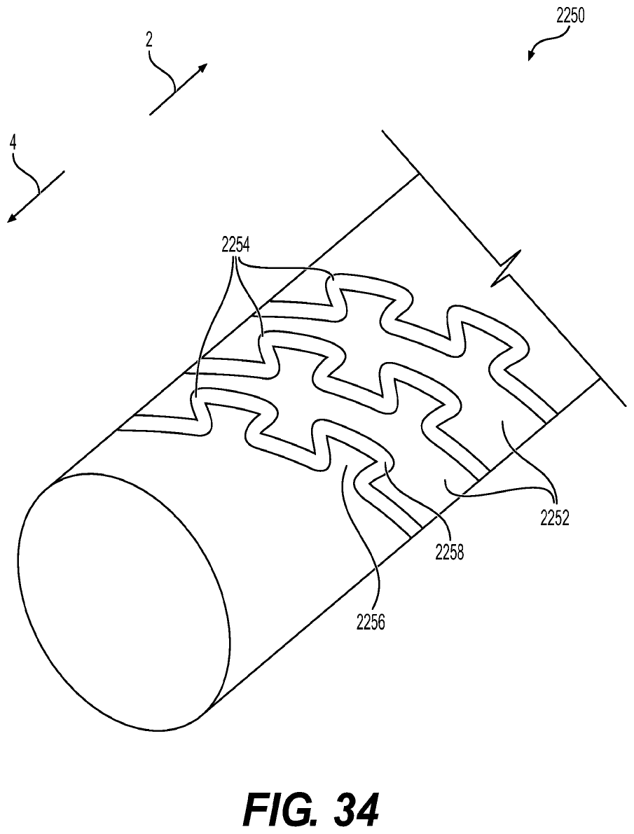

FIG. 34 is a detailed perspective view of a distal end portion of an alternative example structural component 2250 of a shaft of a delivery device, in accordance with at least some aspects of the present disclosure. The structural component 2250 may comprise a laser cut, stainless steel hypotube, generally similar to the structural component 1742 described above with reference to FIGS. 21 and 22. Referring to FIG. 34, in the illustrated embodiment, the solid portions 2252 and/or the gaps 2254 may be arranged in a generally spiral fashion, such as about the longitudinal axis of the structural component 2250. Accordingly, the illustrated portion of the shaft may be flexible in all directions. Additionally, in this example embodiment, the solid portions 2252 and/or gaps 2254 may be formed with respective interlocking features 2256, 2258, such as generally in the form of an interlocking jigsaw pattern. Such a configuration may provide substantial flexibility while still providing desired torque, tension, and/or compression capabilities.

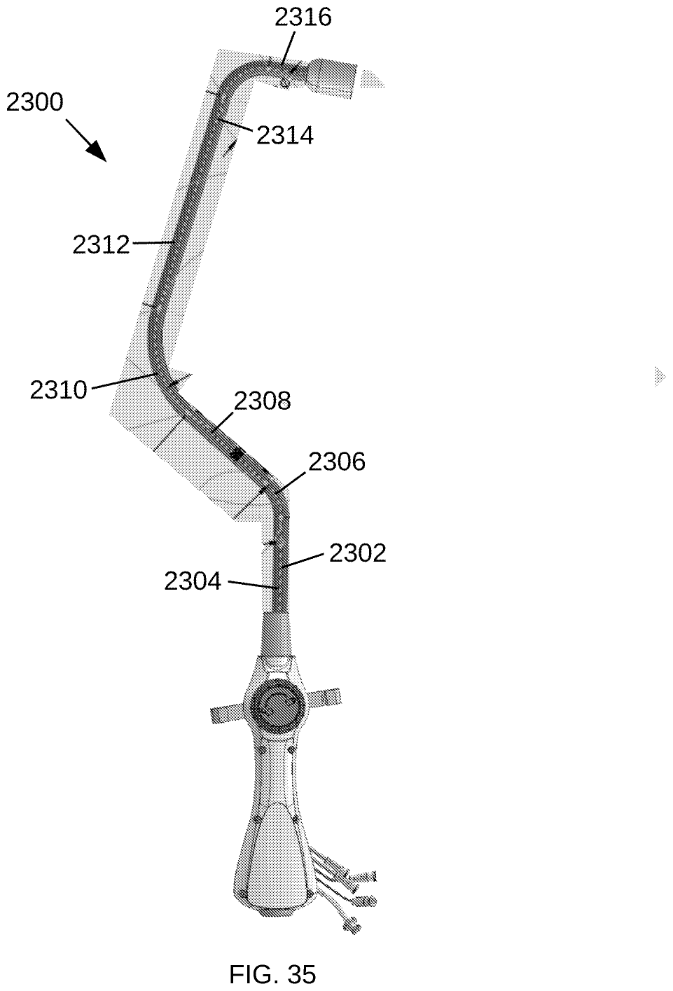

FIG. 35 is a top view of an alternative example delivery device 2300, according to at least some aspects of the present disclosure. The delivery device 2300 is generally similar to the delivery device 1700 of FIG. 17, and repeated description is omitted for brevity. In the illustrated embodiment, the shaft 2302 is configured for multiple bends, such as to facilitate access to a particular surgical site by a therapeutic device delivered therethrough. In the illustrated embodiment, the shaft 2302 is configured with a proximal first generally straight portion 2304, a first bend 2306, a second generally straight portion 2308, a second, opposite bend 2310, a third generally straight portion 2312, a third bend 2314, and a distal fourth generally straight portion 2316. In some example embodiments, various portions of the shaft 2302 may be substantially coplanar. In other example embodiments, one or more of the bends may be in a non-coplanar direction.

Some example embodiments may include more than one inflation lumen. For example, some embodiments may include two (or more) generally parallel inflation lumens that are fluidically connected to an inflatable spacemaker. In embodiments in which both inflation lumens are connected to the same interior chamber of the inflatable spacemaker, such an arrangement may facilitate faster inflation and/or deflation of the inflatable spacemaker by allowing higher inflation fluid flow rates. In other embodiments, the inflatable spacemaker may include more than one fluidically isolated interior chamber. In some such embodiments, one inflation lumen may be fluidically coupled to a first interior chamber and the other inflation lumen may be fluidically coupled a second interior chamber. Accordingly, each interior chamber may be individually inflated and/or deflated, thus facilitating sequential or simultaneous inflation and/or deflation. Additionally, such embodiments may be capable of configurations in which one interior chamber is inflated to a different extent than anther interior chamber. That is, each individual interior chamber may be independently deflated, partially inflated, or fully inflated as desired to configure the inflatable spacemaker in a desired configuration.

Some example embodiments may include more than one suction lumen. For example, some embodiments may include two (or more) generally parallel suction lumens. Some such arrangements may provide redundancy in the event of a partial or full blockage of a suction lumen.

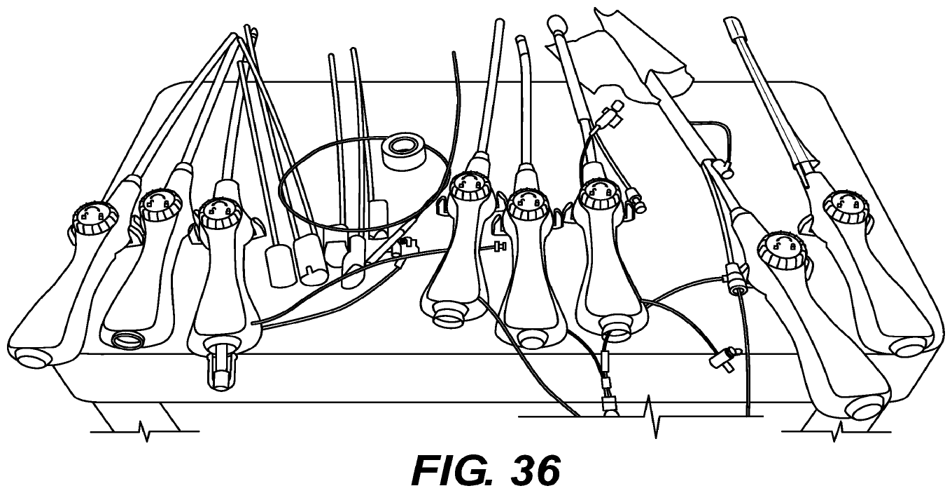

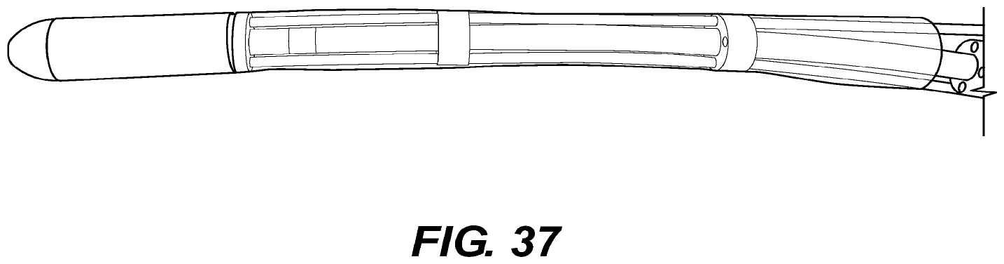

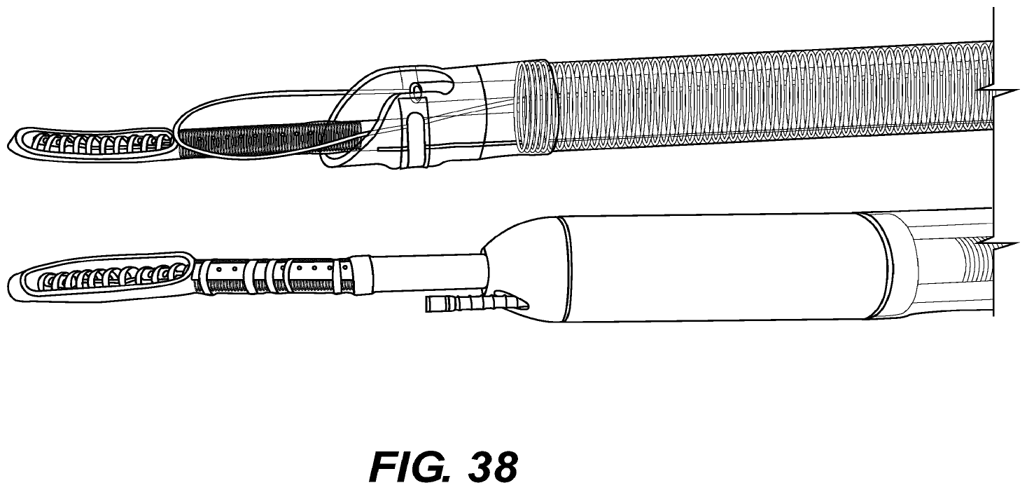

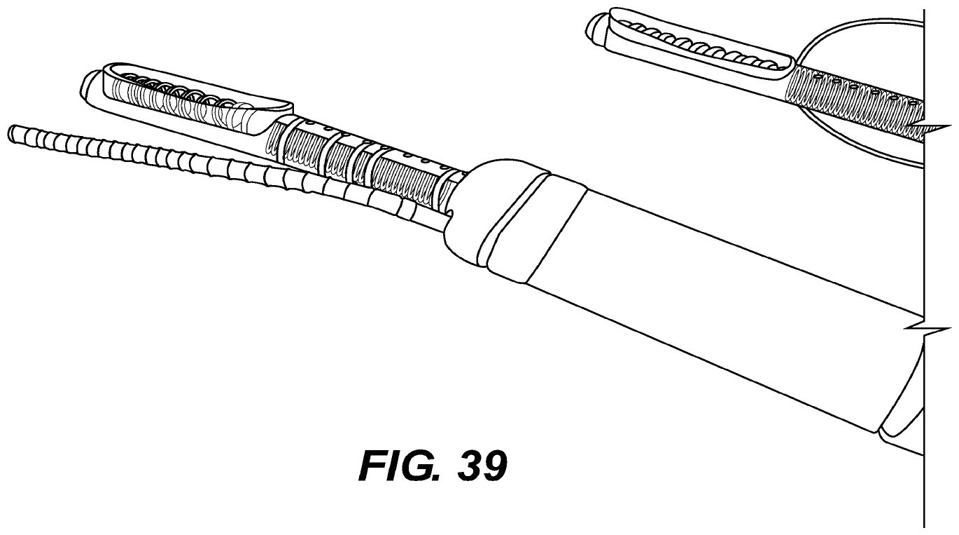

FIGS. 36-39 are perspective view of various alternative example embodiments, all according to at least some aspects of the present disclosure.

Some example methods of using delivery devices according to at least some aspects of the present disclosure may include one or more of the following operations. A distal end portion 1708 of an elongated shaft 1704 of a delivery device 1700 may be positioned at a desired location, such as a surgical site 16. A reconfigurable space-making element 1706 disposed distally on the shaft 1704 may be reconfigured from a retracted configuration to an expanded configuration. A working space 20 may be created or expanded at the desired location using the space-making element 1706. A working end 11 of a therapeutic device 10 may be directed to the desired location through the shaft 1704 via a longitudinally extending therapeutic device lumen 1720.

In some example methods, positioning the distal end portion 1708 of the elongated shaft 1704 of the delivery device 1700 at the desired location may include advancing the shaft 1704 using a handle 1702 disposed proximally on the shaft 1704. The distal end portion 1708 of the shaft 1704 may be steerable by a steering actuator 1758 disposed on the handle 1702. Positioning the distal end portion 1708 of the shaft 1704 of the delivery device 1700 at the desired location may include operating the steering actuator 1758 to steer the distal end portion 1708 of the shaft 1704. Operating the steering actuator 1758 to steer the distal end portion 1708 of the shaft 1704 may include pulling a steering wire 1736, 1738 extending longitudinally through the shaft 1704 to deflect of the distal end portion 1708.

In some example methods, a proximal portion 1709 of the shaft 1704 may be flexible in a first plane 1754. Positioning the distal end portion 1708 of the shaft 1704 of the delivery device 1700 at the desired location may include bending the proximal portion 1709 of the shaft 1704 in the first plane 1754. The distal end portion 1708 of the shaft 1704 is steerable in a second plane 1756, which may be generally perpendicular to the first plane 1754. Positioning the distal end portion 1708 of the shaft 1704 of the delivery device 1700 at the desired location may include steering the distal end portion 1708 of the shaft 1704 in the second plane 1756. The distal end portion 1708 of the shaft 1704 may be flexible in the first plane 1754. Positioning the distal end portion 1708 of the shaft 1704 of the delivery device 1700 at the desired location may include bending the distal end portion 1708 of the shaft 1704 in the first plane 1754.

In some example methods, the reconfigurable space-making element 1706 may include an inflatable space-making element 1706. Expanding the reconfigurable space-making element 1706 may include delivering an inflation fluid to the inflatable space-making element 1706 through a longitudinally disposed inflation lumen 1732, 1734 in the shaft 1704.

In some example methods, expanding the reconfigurable space-making element 1706 may include laterally expanding the reconfigurable space-making element 1706. In some example methods, expanding the reconfigurable space-making element 1706 may include longitudinally expanding the reconfigurable space-making element 1706.

In some example methods, positioning the distal end portion 1708 of the elongated shaft 1704 of the delivery device 1700 at the desired location may include positioning the distal end portion 1708 proximate a left atrial appendage of a patient's heart. Positioning the distal end portion 1708 proximate the left atrial appendage may include advancing the distal end portion 1708 posterior to the patient's heart.

U.S. Pat. No. 8,721,597, issued May 13, 2014, describes apparatus and methods related to diaphragmatic access for surgical access to the heart and is incorporated by reference herein. It is to be understood that various features and operations disclosed therein may be used in connection with any example embodiments according to at least some aspects of the present disclosure. For example, some features and operations pertaining to expandable members disclosed therein may be used in connection with any embodiment according to the present disclosure.

Unless specifically indicated, it will be understood that the description of the structure, function, and/or methodology with respect to any illustrative embodiment herein or any feature or as aspect thereof may apply to any other illustrative embodiments. More generally, it is within the scope of the present disclosure to utilize any one or more features or aspects of any one or more example embodiments described herein in connection with any other one or more features or aspects of any other one or more other example embodiments described herein. Accordingly, any combination of any of the features or aspects of any embodiments described herein is within the scope of this disclosure.

Following from the above description and invention summaries, it should be apparent to those of ordinary skill in the art that, while the methods and apparatuses herein described constitute example embodiments according to the present disclosure, it is to be understood that the scope of the disclosure contained herein is not limited to the above precise embodiments and that changes may be made without departing from the scope of the disclosure. Likewise, it is to be understood that it is not necessary to meet any or all of the identified advantages or objects disclosed herein in order to fall within the scope of the disclosure, since inherent and/or unforeseen advantages may exist even though they may not have been explicitly discussed herein.

What is claimed is:

1. A delivery device configured to deliver a therapeutic device to a surgical site, the delivery device comprising:
a proximally disposed handle;
an elongated shaft extending distally from the handle, the shaft comprising a generally longitudinal therapeutic device lumen configured to deliver a working end of a therapeutic device therethrough, wherein a first portion of the shaft is steerable; and
a space-making element disposed proximate a distal end portion of the shaft and configured to separate biological tissues to create or expand a working space, wherein at least a portion of the space-making element is reconfigurable between a retracted configuration and an expanded configuration.

2. The delivery device of claim 1, wherein the shaft further comprises a generally longitudinal visualization device lumen configured to deliver a working end of a visualization device therethrough.

3. The delivery device of claim 2,
wherein the handle comprises a proximal port operatively connected to the therapeutic device lumen and the visualization device lumen; and
wherein the proximal port is configured to receive the therapeutic device and the visualization device therethrough.

4. The delivery device of claim 1, wherein the shaft further comprises a first generally longitudinal suction lumen configured to facilitate application of suction proximate the distal end portion of the shaft.

5. The delivery device of claim 4, wherein the shaft further comprises a second generally longitudinal suction lumen configured to facilitate application of suction proximate the distal end portion of the shaft.

6. The delivery device of claim 1,
wherein, in the retracted configuration, the space-making element has a width about the same as a width of the shaft; and
wherein, in the expanded configuration, the space-making element has a width substantially greater than the width of the shaft.

7. The delivery device of claim 1, wherein, in the retracted configuration, at least a portion of the space-making element is disposed generally concentrically within the distal end portion of the shaft.

8. The delivery device of claim 7, wherein the distal end portion of the shaft comprises a recess configured to receive at least a portion of the space-making element in the retracted configuration.

9. The delivery device of claim 1, wherein the space-making element comprises an inflatable space-making element.

10. The delivery device of claim 9, wherein the shaft comprises a first generally longitudinal inflation lumen operatively coupled to supply inflation fluid to the inflatable space-making element.

11. The delivery device of claim 9, wherein the inflatable space-making element is generally in the form of a right circular cylinder.

12. The delivery device of claim 9, wherein the inflatable space-making element comprises a plurality of generally circumferentially oriented ribs.

13. The delivery device of claim 9, wherein the inflatable space-making element comprises a plurality of generally longitudinally oriented ribs.

14. The delivery device of claim 9, wherein the inflatable space-making element comprises at least one orientation feature.

15. The delivery device of claim 14, wherein the orientation feature comprises a notch.

16. The delivery device of claim 9, wherein the inflatable space-making element comprises a distal face that is angled relative to a plane normal to a longitudinal axis of the shaft.

17. The delivery device of claim 9, wherein the inflatable space-making element comprises a plurality of longitudinally oriented inflatable elements.

18. The delivery device of claim 17, wherein at least one of the plurality of longitudinally oriented inflatable elements is in the form of a generally circular tube.

19. The delivery device of claim 17, wherein at least one of the plurality of longitudinally oriented inflatable elements is in the form of a tube having a generally oval cross section.

20. The delivery device of claim 17, wherein the inflatable space-making element comprises at least one collapsible structural element configured to facilitate spacing of the longitudinally oriented inflatable elements.

21. The delivery device of claim 20, wherein the collapsible structural element comprises a collapsible nitinol frame.

22. The delivery device of claim 9, wherein the inflatable space-making element comprises at least one generally helical inflatable element.

23. The delivery device of claim 22, wherein the inflatable space-making element comprises two generally helical inflatable elements in an alternating arrangement.

24. The delivery device of claim 1,
wherein the first portion of the shaft is steerable in a first plane; and
wherein the handle comprises a steering actuator configured to steer the first portion of the shaft in the first plane.

25. The delivery device of claim 24, wherein the first portion of the shaft is steerable uni-directionally in the first plane.

26. The delivery device of claim 24, wherein the first portion of the shaft is steerable bi-directionally in the first plane.

27. The delivery device of claim 24, wherein the first portion of the shaft is deformable in a second plane, the second plane generally perpendicular to the first plane.

28. The delivery device of claim 27, wherein the first portion of the shaft is flexible in the second plane.

29. The delivery device of claim 27, wherein the first portion of the shaft is steerable in the second plane.

30. The delivery device of claim 29, wherein the first portion of the shaft is steerable bi-directionally in the first plane and bi-directionally in the second plane.

31. The delivery device of claim 24, further comprising a first steering wire operatively coupling the steering actuator and the first portion of the shaft for steering the first portion of the shaft a first direction in the first plane.

32. The delivery device of claim 31, further comprising a second steering wire operatively coupling the steering actuator and the first portion of the shaft for steering the first portion of the shaft in a second direction in the first plane, the second direction generally opposite the first direction.

33. The delivery device of claim 24, wherein a second portion of the shaft is flexible in a second plane, the second plane generally perpendicular to the first plane.

34. The delivery device of claim 24, wherein the handle further comprises at least one locking actuator configured to selectively lock the steering actuator.

35. The delivery device of claim 1, wherein a second portion of the shaft is flexible in a first plane.

36. The delivery device of claim 35, wherein the second portion of the shaft is flexible in a second plane, the second plane generally perpendicular to the first plane.

37. The delivery device of claim 36, wherein a flexibility of the second portion of the shaft in the first plane is greater than a flexibility of the second portion of the shaft in the second plane.

38. The delivery device of claim 36, wherein a stiffness of the second portion of the shaft in the first plane is greater than a stiffness of the second portion of the shaft in the second plane.

39. The delivery device of claim 35, wherein the second portion of the shaft is substantially uniformly flexible over a longitudinal length of the second portion of the shaft.

40. The delivery device of claim 35, wherein a shaft flexibility varies longitudinally over a longitudinal length of the second portion of the shaft.

41. The delivery device of claim 35, wherein a flexibility of the first portion of the shaft differs from a flexibility of the second portion of the shaft.

42. The delivery device of claim 1, wherein the shaft further comprises an outer jacket and a structural component disposed radially within the outer jacket.

43. The delivery device of claim 42, wherein the structural component comprises a hypotube.

44. The delivery device of claim 43, wherein the hypotube comprises a plurality of solid portions and a plurality of gaps configured to facilitate desired flexibility characteristics of the shaft.

45. The delivery device of claim 44, wherein the hypotube comprises a first arrangement of the solid portions and the gaps in the first portion of the shaft and a second arrangement of the solid portions and the gaps in a second portion of the shaft so that the first portion of the shaft and the second portion of the shaft have different flexibility characteristics.

46. The delivery device of claim 1, further comprising an obturator positioned distally on the shaft.

47. The delivery device of claim 46, wherein the obturator is inflatable.

48. The delivery device of claim 46, wherein the obturator is selectively removable from the delivery device.

49. The delivery device of claim 1, wherein the therapeutic device comprises at least one of an ablation device, a biopsy device, a pericardial lead, a needle for delivering a therapeutic agent, and a surgical tool.

50. The delivery device of claim 1, wherein the visualization device comprises an endoscope.

51. A delivery device configured to deliver a therapeutic device to a surgical site, the delivery device comprising:
a proximally disposed handle;
an elongated shaft extending from the handle, the shaft comprising a generally longitudinal therapeutic device lumen configured to deliver a working end of a therapeutic device therethrough, wherein a first portion of the shaft is flexible; and
a space-making element disposed proximate a distal end portion of the shaft and configured to separate biological tissues to create or expand a working space.

52. The delivery device of claim 51, wherein the space-making element is reconfigurable between a retracted configuration and an expanded configuration by inflation and deflation of at least one inflatable element.

53. The delivery device of claim 51, wherein the space-making element is substantially rigid.

54. The delivery device of claim 53, wherein the space-making element comprises a deep, angled hood.

55. The delivery device of claim 51, wherein the first portion of the shaft is flexible in a first plane.

56. The delivery device of claim 55, wherein a second portion of the shaft is flexible in a second plane, the second plane generally perpendicular to the first plane.

57. The delivery device of claim 56,
wherein the first portion of the shaft comprises a distal portion; and
wherein the second portion of the shaft comprises a proximal portion.

58. The delivery device of claim 57, wherein the first portion of the shaft is steerable in the first plane by operation of a steering actuator disposed on the handle.

59. The delivery device of claim 56, wherein the first portion of the shaft is flexible in the second plane.

60. A method of using a delivery device, comprising:
positioning a distal end portion of an elongated shaft of a delivery device at a desired location;
expanding a reconfigurable space-making element disposed distally on the shaft from a retracted configuration to an expanded configuration;
creating or expanding a working space at the desired location using the space-making element; and
directing a working end of a therapeutic device to the desired location through the shaft via a longitudinally extending therapeutic device lumen.

61. The method of claim 60, wherein positioning the distal end portion of the elongated shaft of the delivery device at the desired location comprises advancing the shaft using a handle disposed proximally on the shaft.

62. The method of claim 61,
wherein the distal end portion of the shaft is steerable by a steering actuator disposed on the handle; and
wherein positioning the distal end portion of the shaft of the delivery device at the desired location comprises operating the steering actuator to steer the distal end portion of the shaft.

63. The method of claim 62, wherein operating the steering actuator to steer the distal end portion of the shaft comprises pulling a steering wire extending longitudinally through the shaft to deflect of the distal end portion.

64. The method of claim 60,
wherein a proximal portion of the shaft is flexible in a first plane; and
wherein positioning the distal end portion of the shaft of the delivery device at the desired location comprises bending the proximal portion of the shaft in the first plane.

65. The method of claim 64,
wherein the distal end portion of the shaft is steerable in a second plane, the second plane generally perpendicular to the first plane; and
wherein positioning the distal end portion of the shaft of the delivery device at the desired location comprises steering the distal end portion of the shaft in the second plane.

66. The method of claim 65,
wherein the distal end portion of the shaft is flexible in the first plane; and
wherein positioning the distal end portion of the shaft of the delivery device at the desired location comprises bending the distal end portion of the shaft in the first plane.

67. The method of claim 60,
wherein the reconfigurable space-making element comprises an inflatable space-making element; and
wherein expanding the reconfigurable space-making element comprises delivering an inflation fluid to the inflatable space-making element through a longitudinally disposed inflation lumen in the shaft.

68. The method of claim 60, wherein expanding the reconfigurable space-making element comprises laterally expanding the reconfigurable space-making element.

69. The method of claim 60, wherein expanding the reconfigurable space-making element comprises longitudinally expanding the reconfigurable space-making element.

70. The method of claim 60, wherein positioning the distal end portion of the elongated shaft of the delivery device at the desired location comprises positioning the distal end portion proximate a left atrial appendage of a patient's heart.

71. The method of claim 70, wherein positioning the distal end portion proximate the left atrial appendage comprises advancing the distal end portion posterior to the patient's heart.

72. Any apparatus, method, or combination thereof as disclosed herein.

73. Any two or more of the foregoing claims in any combination.

74. Any combination of elements from one or more of the foregoing claims.
